(19) United States
(12) Patent Application Publication
(10) Pub. No.: US 2026/0027123 A1
(43) Pub. Date: Jan. 29, 2026

(54) Compositions for Modulating Lipoxygenase and Methods of Using Same

(71) Applicant: The Regents of the University of California, Oakland, CA (US)

(72) Inventor: Theodore HOLMAN, Santa Cruz, CA (US)

(21) Appl. No.: 18/992,933

(22) PCT Filed: Jul. 17, 2023
(86) PCT No.: PCT/US2023/027879

§ 371 Date: Jan. 9, 2025

Related U.S. Application Data
(60) Provisional application No. 63/390,236, filed on Jul. 18, 2022.

(51) Int. Cl. A61K 31/5377; A61K 31/421; A61K 31/426; A61K 31/428; A61K 31/429; A61K 31/437; A61K 31/4439; A61K 31/4985; A61K 47/10; A61K 47/14; A61K 47/18; A61K 47/44

(52) U.S. Cl. CPC A61K 31/5377; A61K 31/421; A61K 31/426; A61K 31/428; A61K 31/429; A61K 31/437; A61K 31/4439; A61K 31/4985; A61K 47/10; A61K 47/14; A61K 47/18; A61K 47/44

(57) ABSTRACT

Compositions for inhibiting lipoxygenase enzymes (e.g., human epithelial 12/15-lipoxygenase (12/15-LOX) are provided. Compositions according to certain embodiments include a first solvent, a second solvent and a lipoxygenase inhibitor. In certain instances, the lipoxygenase inhibitor is present in the composition (e.g., compositions formulated for intravenous administration) at a concentration of 30 mg/mL or greater. Methods for treating or preventing a lipoxygenase (e.g., human epithelial 12/15-lipoxygenase (12/15-LOX))-mediated disease or disorder are also provided.

### CROSS REFERENCE TO RELATED APPLICATIONS

Pursuant to 35 U.S.C. § 119(e), this application claims priority to the filing date of U.S. Provisional Patent Application Ser. No. 63/390,236 filed on Jul. 18, 2022, the disclosure of which application is incorporated herein by reference in its entirety.

### GOVERNMENT RIGHTS

This invention was made with Government support under Grant No. NS106854, awarded by the National Institutes of Health (NIH). The Government has certain rights in the invention.

### INTRODUCTION

Lipoxygenases catalyze the peroxidation of fatty acids which contain bisallylic hydrogens between two cis double bonds, such as in linoleic acid (LA) and arachidonic acid (AA).

Lipoxygenases are named according to their product specificity with AA as the substrate because AA is the precursor of many active lipid metabolites that are involved in a number of significant disease states. The human genome contains six functional human lipoxygenases (LOX) genes (ALOX5, ALOX12, ALOX12B, ALOX15, ALOX15B, eLOX3) encoding for six different human LOX isoforms (h5-LOX, h12S-LOX, h12R-LOX, h15-LOX-1, h15-LOX-2, eLOX3, respectively). The biological role in health and disease for each LOX isozyme varies dramatically, ranging from asthma to diabetes or stroke.

The nomenclature of the LOX isozymes is loosely based on the carbon position (e.g., 5, 12, or 15) at which they oxidize arachidonic acid to form the corresponding hydroperoxyeicosatetraenoic acid (HpETE), which is reduced to the hydroxyeicosatetraenoic acid (HETE) by intracellular glutathione peroxidases.

Lipoxygenase inhibitors are difficult to formulate due to challenges with solubility and other factors, therefore new formulations are needed.

### SUMMARY

Compositions for inhibiting lipoxygenase enzymes (e.g., human reticulocyte 12/15-lipoxygenase (12/15-LOX)) are provided. Compositions according to certain embodiments include a first solvent, a second solvent and a lipoxygenase inhibitor. In certain instances, the lipoxygenase inhibitor is present in the composition (e.g., compositions formulated for intravenous administration) at a concentration of 30 mg/mL or greater. Methods for treating or preventing a lipoxygenase (e.g., human reticulocyte 12/15-lipoxygenase (12/15-LOX))-mediated disease or disorder are also provided.

In some embodiments, the first solvent includes a solvent selected from dimethylacetamide (DMA), diethylacetamide (DEA), dimethylsulfoxide (DMSO), a polyoxyethylene ester of 12-hydroxysteraric acid, a polyethoxylated castor oil, a polyvinyl caprolactam-polyvinyl acetate-polyethylene glycol, a propylene glycol caprylate, a diethylene glycol monomethyl ether, a caprylocaproyl macrogolglyceride, a polyoxyl-6 glyceride and a propylene glycol monolaurate. In certain instances, the polyoxyethylene ester of 12-hydroxysteraric acid includes a solvent that is or is substantially the same as Solutol. In certain instances, the polyethoxylated castor oil includes a solvent that is or is substantially the same as Kolliphor. In certain instances, the solvent includes a solvent that is or is substantially the same as Kolliphor EL. In certain instances, the solvent includes a solvent that is or is substantially the same as Kolliphor RH40. In certain instances, the solvent includes a solvent that is or is substantially the same as Kolliphor P188 Geismar. In certain instances, the polyvinyl caprolactam-polyvinyl acetate-polyethylene glycol includes a solvent that is or is substantially the same as Soluplus. In certain instances, the propylene glycol caprylate includes a solvent that is or is substantially the same as Capryol. In certain instances, the diethylene glycol monomethyl ether includes a solvent that is or is substantially the same as Transcutol. In certain instances, the caprylocaproyl macrogolglyceride includes a solvent that is or is substantially the same as Labrasol. In certain instances, the polyoxyl-6 glyceride includes a solvent that is or is substantially the same as Labrafil. In certain instances, the propylene glycol monolaurate includes a solvent that is or is substantially the same as Lauroglycol. In some embodiments, the second solvent includes an alcohol solvent, such as ethanol. In some instances, the second solvent includes a polyethylene glycol. In certain instances, the second solvent includes polyethylene glycol 400.

The amount of first solvent and second solvent present in the composition may range, such as in a weight ratio of from 1:99 to 99:1, such as from 2:98 to 98:2, such as from 3:97 to 97:3, such as from 4:96 to 96:4, such as from 5:95 to 95:5, such as from 6:94 to 94:6, such as from 7:93 to 93:7, such as from 8:92 to 92:8, such as from 9:91 to 91:9, such as from 10:90 to 90:10, such as from 20:80 to 80:20, such as from 30:70 to 70:30, such as from 40:60 to 60:40 and including a weight ratio of the first solvent to second solvent of 50:50. In certain instances, the volume ratio of first solvent to second solvent ranges from 1:99 to 99:1, such as from 2:98 to 98:2, such as from 3:97 to 97:3, such as from 4:96 to 96:4, such as from 5:95 to 95:5, such as from 6:94 to 94:6, such as from 7:93 to 93:7, such as from 8:92 to 92:8, such as from 9:91 to 91:9, such as from 10:90 to 90:10, such as from 20:80 to 80:20, such as from 30:70 to 70:30, such as from 40:60 to 60:40 and including a volume ratio of the first solvent to second solvent of 50:50. In certain instances, the molar ratio of first solvent to second solvent ranges from 1:99 to 99:1, such as from 2:98 to 98:2, such as from 3:97 to 97:3, such as from 4:96 to 96:4, such as from 5:95 to 95:5, such as from 6:94 to 94:6, such as from 7:93 to 93:7, such as from 8:92 to 92:8, such as from 9:91 to 91:9, such as from 10:90 to 90:10, such as from 20:80 to 80:20, such as from 30:70 to 70:30, such as from 40:60 to 60:40 and including a molar ratio of the first solvent to second solvent of 50:50.

In some embodiments, the composition is formulated for intravenous administration to a subject, such as by bolus injection or by sustained infusion. In certain instances, the composition is formulated such that the concentration of the lipoxygenase inhibitor present in the composition is 25 mg/mL or more, such as 30 mg/mL or more, such as 35 mg/mL or more, such as 40 mg/mL or more, such as 45 mg/mL or more, such as 50 mg/mL or more, such as 55 mg/mL or more, such as 60 mg/mL or more, such as 65 mg/mL or more, such as 70 mg/mL or more, such as 75 mg/mL or more, such as 80 mg/mL or more, such as 85 mg/mL or more, such as 90 mg/mL or more, such as 95 mg/mL or more, such as 100 mg/mL or more, such as 110 mg/mL or more, such as 120 mg/mL or more, such as 130 mg/mL or more, such as 140 mg/mL or more, such as 150 mg/mL or more, such as 175 mg/mL or more, such as 200 mg/mL or more and including 250 mg/mL or more.

In some embodiments, the lipoxygenase inhibitor present in compositions of interest is a compound of Formula (I):

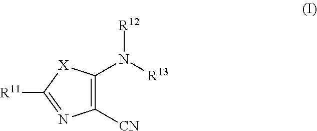

or a pharmaceutically acceptable salt thereof, in which:
X is O or S;
R¹¹ is an aryl, heteroaryl, cyclyl, or heterocyclyl, each of which can be optionally substituted; and
R¹² and R¹³ are independently hydrogen, halogen, alkyl, alkenyl, alkynyl, aralkyl, acyl, aryl, heteroaryl, cyclyl or heterocyclyl, each of which can be optionally substituted or a pharmaceutically acceptable salt thereof.

In some embodiments, the lipoxygenase inhibitor present in compositions of interest is a compound of Formula (Ia):

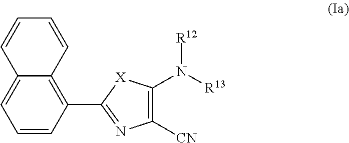

or a pharmaceutically acceptable salt thereof, in which:
X is O or S;
R¹² and R¹³ are independently hydrogen, halogen, alkyl, alkenyl, alkynyl, aralkyl, acyl, aryl, heteroaryl, cyclyl or heterocyclyl, each of which can be optionally substituted
or a pharmaceutically acceptable salt thereof.

In certain instances, the lipoxygenase inhibitor is selected from 5-(methylamino)-2-naphthalen-1-yl-1,3-oxazole-4-carbonitrile (ML351), 2-(2,3-dichlorophenyl)-5-(methylamino)-1,3-oxazole-4-carbonitrile, 2-(3,4-dichlorophenyl)-5-(methylamino)-1,3-oxazole-4-carbonitrile, 5-(methylamino)-2-naphthalen-1-yl-1,3-thiazole-4-carbonitrile or a pharmaceutically acceptable salt thereof.

In some embodiments, the lipoxygenase inhibitor present in compositions of interest is a a compound of Formula (II):

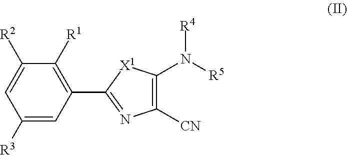

or a pharmaceutically acceptable salt thereof, wherein:
X¹ is selected from O and S;
R¹, R², and R³ are each independently selected from halo, CN, C₁₋₃ alkyl, C₁₋₃ haloalkyl, C₁₋₃ alkoxy, and C₁₋₃ haloalkoxy;
R⁴ is selected from H, C₁₋₃ alkyl, and HO—C₁₋₃ alkylene;
R⁵ is selected from C₁₋₆ alkyl, C₂₋₆ alkenyl, C₂₋₆ alkynyl, C(O)OR^{a1},
C(O)N(R^{a1})₂, P(═O)(OR^{a1})₂, and C(O)R^{b1}; wherein said C₁₋₆ alkyl, C₂₋₆ alkenyl, and C₂₋₆ alkynyl are each optionally substituted with a substituent selected from OR^{a1} and OP(═O)(OR^{a1})₂;
each R^{a1} is independently selected from H, C₁₋₆ alkyl, C₆₋₁₀ aryl, C₁₋₆ alkyl-C₆₋₁₀ aryl, and C₁₋₆ alkyl-C₆₋₁₀ aryl-C₁₋₆ alkyl, wherein said C₁₋₆ alkyl, C₆₋₁₀ aryl, C₁₋₆ alkyl-C₆₋₁₀ aryl, and C₁₋₆ alkyl-C₆₋₁₀ aryl-C₁₋₆ alkyl are each optionally substituted with a substituent selected from amino, C₁₋₆ alkylamino, (C₁₋₆ haloalkyl)amino, di(C₁₋₆ alkyl)amino, (C₁₋₆ alkyl)(C₁₋₆ haloalkyl)amino, (C₆₋₁₀ aryl)amino, (C₆₋₁₀ aryl)(C₁₋₆ alkyl)amino, (5-6-membered heteroaryl)amino, (5-6-membered heteroaryl)(C₁₋₆ alkyl)amino, C₆₋₁₀ aryl, 4-6 membered heterocycloalkyl, 5-6-membered heteroaryl, and OR^{a2}, wherein said C₆₋₁₀ aryl, 4-6 membered heterocycloalkyl, and 5-6-membered heteroaryl are each optionally substituted with 1, 2, or 3 substituents independently selected from amino, C₁₋₆ alkylamino, di(C₁₋₆ alkyl)amino, carboxy, and halo;
each R^{a2} is independently selected from H, C₁₋₃ alkyl, C₁₋₃ haloalkyl, C₁₋₃ alkoxy-C₁₋₃ alkyl, 4-7 membered heterocycloalkyl-C₁₋₃ alkyl, 5-6-membered heteroaryloxy-C₁₋₃ alkyl, C₆₋₁₀ aryl, and 5-6-membered heteroaryl, wherein said C₆₋₁₀ aryl and 5-6-membered heteroaryl are each optionally substituted with 1, 2, or 3 substituents independently selected from halo, C₁₋₃ alkoxy, C₁₋₃ haloalkoxy, C₁₋₃ alkyl, and C₁₋₃ haloalkyl; and
R^{b1} is C₁₋₆ alkyl, optionally substituted with a substituent selected from amino, C₁₋₆ alkylamino, di(C₁₋₆ alkyl)amino, and 4-7 membered heterocycloalkyl ring comprising at least one N atom
or a pharmaceutically acceptable salt thereof.

In certain embodiments, the lipoxygenase inhibitor is a compound selected from:

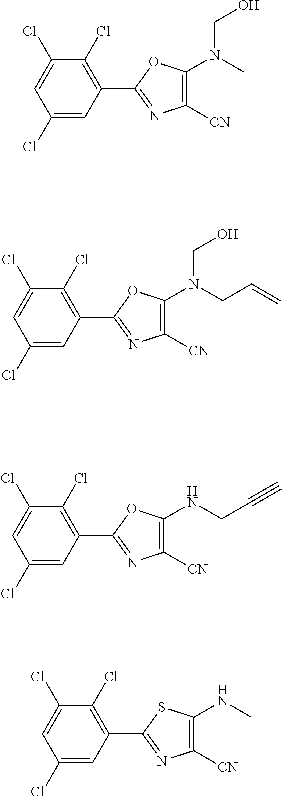 [SMILES: CN(CO)c1oc(-c2cc(Cl)cc(Cl)c2Cl)nc1C#N ; C=CCN(CO)c1oc(-c2cc(Cl)cc(Cl)c2Cl)nc1C#N ; C#CCNc1oc(-c2cc(Cl)cc(Cl)c2Cl)nc1C#N ; CNc1sc(-c2cc(Cl)cc(Cl)c2Cl)nc1C#N]

or a pharmaceutically acceptable salt thereof.

In some embodiments, the lipoxygenase inhibitor in compositions of interest is a compound of formula of Formula (III):

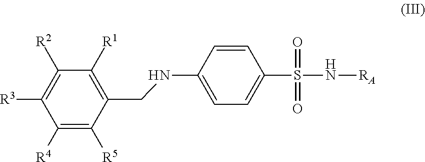

where R¹, R², R³, R⁴ and R⁵ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
R_{A} is cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl,
or a salt, solvate or hydrate thereof.

In some embodiments, R¹ is hydroxy. In some embodiments, R² is alkoxy. In some instances, R² is a C₁-C₁₂ alkoxy. In some instances, R² is selected from methoxy, ethoxy, propoxy, butoxy, isobutoxy and tert-butoxy. In certain instances, R² is methoxy. In some instances, R³ is hydrogen. In some instances, R⁴ is hydrogen. In some instances, R⁵ is hydrogen. In some instances, each of R³, R⁴ and R⁵ are hydrogen.

In some embodiments, R_{A} is selected from the group consisting of 2-benzothiazole, 2-benzoxazole, 2-benzimidazole, 2-thiophene, 4-methyl-2-benzothiazole, 4-methyl-2-thiazole, 5-methyl-2-thiazole, 5-phenyl-2-thiazole, 4,5-methyl-2-thiazole, 5-methyl-3-isoxazole, 3-methoxy-phenyl, 3-quinolone, 8-isoquinolone, phenyl, 1-naphthalene, 2-naphthalene, 1,4-bi-phenyl, 1.3-bi-phenyl, 3-piperazine-phenyl, 4-piperazine-phenyl, 4-piperidine-phenyl, 4-piperazine-3-pyridine, 6-methyl-3-pyridine, 2-pyridine, 3-pyridine, 2-pyrimidine, 3-tert-butyl-phenyl, 6-methoxy-2-benzothiazole, 4-phenyl-2-thiazole, 3-morpholine-phenyl, 4N-boc-piperidine-3-phenyl, 3-piperidine-phenyl, 3-isopropyl-phenyl and 6-F-2-benzothiazole.

In some embodiments, the lipoxygenase inhibitor in compositions of interest is a compound of formula of Formula (IIIA):

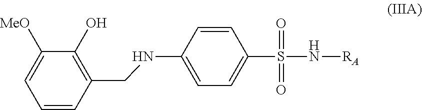

where R_{A} is selected from the group consisting of 2-benzothiazole, 2-benzoxazole, 2-benzimidazole, 2-thiophene, 4-methyl-2-benzothiazole, 4-methyl-2-thiazole, 5-methyl-2-thiazole, 5-phenyl-2-thiazole, 4,5-methyl-2-thiazole, 5-methyl-3-isoxazole, 3-methoxy-phenyl, 3-quinolone, 8-isoquinolone, phenyl, 1-naphthalene, 2-naphthalene, 1,4-bi-phenyl, 1.3-bi-phenyl, 3-piperazine-phenyl, 4-piperazine-phenyl, 4-piperidine-phenyl, 4-piperazine-3-pyridine, 6-methyl-3-pyridine, 2-pyridine, 3-pyridine, 2-pyrimidine, 3-tert-butyl-phenyl, 6-methoxy-2-benzothiazole, 4-phenyl-2-thiazole, 3-morpholine-phenyl, 4N-boc-piperidine-3-phenyl, 3-piperidine-phenyl, 3-isopropyl-phenyl and 6-F-2-benzothiazole.

In certain embodiments, the lipoxygenase inhibitor in compositions of interest is a compound of formula of Formula (IIIB):

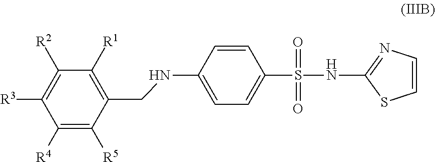

where R¹, R², R³, R⁴ and R⁵ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl.

In some embodiments, R¹, R², R³, R⁴ and R⁵ are each hydrogen. In some embodiments, R¹ is hydroxy. In some embodiments, R² is alkoxy. In some embodiments, R³ is alkoxy. In some instances, R² is a C₁-C₁₂ alkoxy. In some instances, R² is selected from methoxy, ethoxy, propoxy, butoxy, isobutoxy and tert-butoxy. In certain instances, R² is methoxy. In some instances, R³ is hydrogen. In some instances, R³ is a C₁-C₁₂ alkoxy. In some instances, R³ is selected from methoxy, ethoxy, propoxy, butoxy, isobutoxy and tert-butoxy. In certain instances, R³ is methoxy. In some instances, R⁴ is hydrogen. In some instances, R⁴ is a C₁-C₁₂ alkoxy. In some instances, R⁴ is selected from methoxy, ethoxy, propoxy, butoxy, isobutoxy and tert-butoxy. In certain instances, R⁴ is methoxy. In some instances, R⁵ is hydrogen. In some instances, each of R³, R⁴ and R⁵ are hydrogen. In some embodiments, R¹, R², R³, R⁴ and R⁵ are each independently selected from a halogen such as Cl, F or Br, methyl, amino, nitro, allyl and methoxy.

In certain embodiments, the lipoxygenase inhibitor in compositions of interest is a compound of formula of Formula (IIIC):

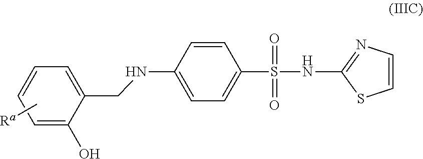

where R^{a} is selected from 2-amino-Ph; 3-OH-Ph; 2-amino-3-methoxy-Ph; 3-OH-4-methoxy-Ph; 7-indole; 2,3-Cl-Ph; 3-Cl; 3-F; 3-Br; 4-Br; 3-methyl; 3-amino; 3-nitro; 3-allyl; 4-Cl; 4-methoxy; 5-Cl; 5-methoxy; 5-nitro; 5-F; 5-nitro and 6-methoxy.

In certain embodiments, the lipoxygenase inhibitor in compositions of interest is a compound of formula of Formula (IIID):

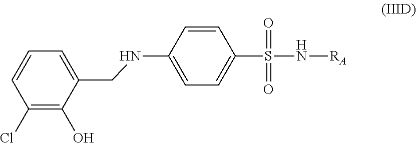

where R_{A} is selected from the group consisting of phenyl, 1-naphthalene, 2-benzothiazole, 2-naphthalene, 4-biphenyl, 8-isoquinoline, 3-quinoline and 4-piperidine-phenyl.

In certain embodiments, the lipoxygenase inhibitor in compositions of interest is a compound of formula of Formula (IIIE):

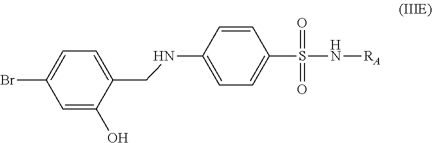

where R_{A} is selected from the group consisting of phenyl, 1-naphthalene, 2-benzothiazole, 3-quinoline, 2-naphthalene, 4-biphenyl and 8-quinoline.

In certain embodiments, the lipoxygenase inhibitor in compositions of interest is a compound of formula of Formula (IIIF):

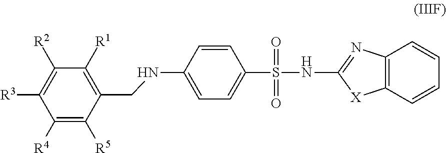

where R¹, R², R³, R⁴ and R⁵ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
X is S or O
or a salt, solvate or hydrate thereof.

In some embodiments, R¹, R², R³, R⁴ and R⁵ are each hydrogen. In some embodiments, R¹ is hydroxy. In some embodiments, R² is alkoxy. In some embodiments, R³ is alkoxy. In some instances, R² is a C₁-C₁₂ alkoxy. In some instances, R² is selected from methoxy, ethoxy, propoxy, butoxy, isobutoxy and tert-butoxy. In certain instances, R² is methoxy. In some instances, R³ is hydrogen. In some instances, R³ is a C₁-C₁₂ alkoxy. In some instances, R³ is selected from methoxy, ethoxy, propoxy, butoxy, isobutoxy and tert-butoxy. In certain instances, R³ is methoxy. In some instances, R⁴ is hydrogen. In some instances, R⁴ is a C₁-C₁₂ alkoxy. In some instances, R⁴ is selected from methoxy, ethoxy, propoxy, butoxy, isobutoxy and tert-butoxy. In certain instances, R⁴ is methoxy. In some instances, R⁵ is hydrogen. In some instances, each of R³, R⁴ and R⁵ are hydrogen. In some embodiments, R¹, R², R³, R⁴ and R⁵ are each independently selected from a halogen such as Cl, F or Br, methyl, amino, nitro, allyl and methoxy.

In some embodiments, X is oxygen. In other embodiments, X is S.

In certain embodiments, the lipoxygenase inhibitor in compositions of interest is a compound of formula of Formula (IIIG):

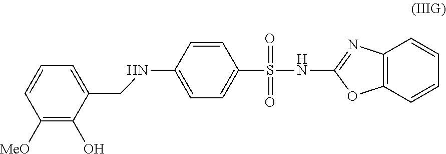 [SMILES: COc1cccc(CNc2ccc(S(=O)(=O)Nc3nc4ccccc4o3)cc2)c1O]

In certain embodiments, the lipoxygenase inhibitor in compositions of interest is a compound of formula of Formula (IIIH):

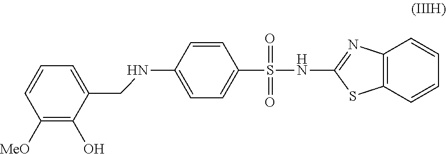 [SMILES: COc1cccc(CNc2ccc(S(=O)(=O)Nc3nc4ccccc4s3)cc2)c1O]

In some embodiments, the lipoxygenase inhibitor in compositions of interest is a compound of formula of Formula (IV):

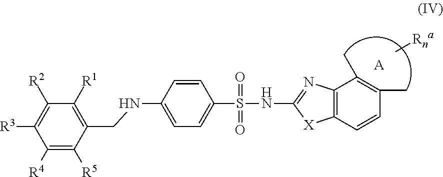

where R¹, R², R³, R⁴ and R⁵ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
X is S or O;
the A ring is absent or when the A ring is present, the A ring is a substituted or unsubstituted 5 to 12 membered ring;
n is an integer from 0 to 12; and
each R^{a} is independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl,
or a salt, solvate or hydrate thereof.

In some embodiments, R¹ is hydroxy. In some embodiments, R² is alkoxy. In some instances, R² is a C₁-C₁₂ alkoxy. In some instances, R² is selected from methoxy, ethoxy, propoxy, butoxy, isobutoxy and tert-butoxy. In certain instances, R² is methoxy. In some instances, R³ is hydrogen. In some instances, R⁴ is hydrogen. In some instances, R⁵ is hydrogen. In some instances, each of R³, R⁴ and R⁵ are hydrogen.

In some embodiments, X is oxygen. In other embodiments, X is S.

In some embodiments, A is a six-membered ring. In some instances, A is a six-membered heterocyclic ring. In some instances, A is a six-membered aryl ring. In some instances, A is a six-membered heteroaryl ring. In some embodiments, A is a six-membered ring and the compound is of formula (IA):

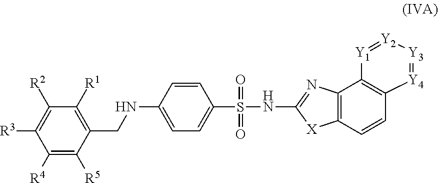

where Y₁, Y₂, Y₃ and Y₄ are each independently C or N. In some embodiments, Y₁, Y₂, Y₃ and Y₄ are each C. In some embodiments, Y₃ is N and Y₁, Y₂, and Y₄ are each C. In some embodiments, Y₁ and Y₄ are N; and Y₂ and Y₃ are C. In some embodiments, A is unsubstituted.

In some embodiments, A is monosubstituted. In other embodiments, A is di-substituted. In other embodiments, A is tri-substituted. In some instances, n is 1 or 2 and each R^{a} is independently selected from:

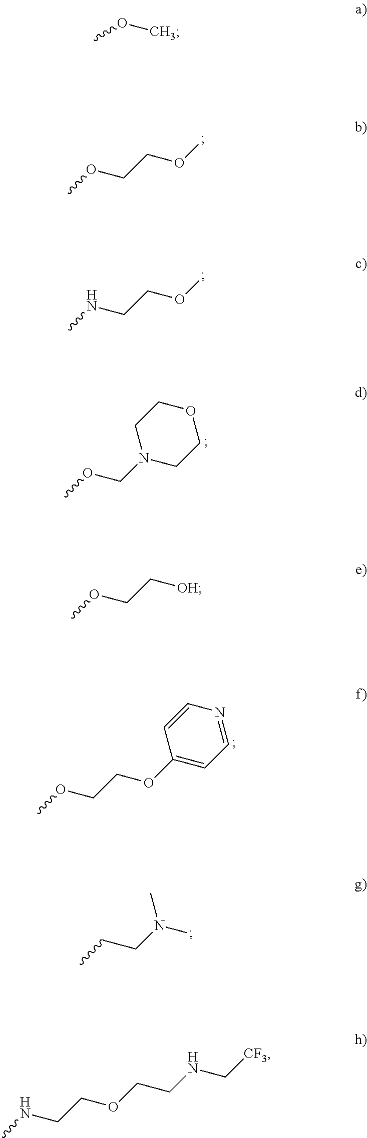

where [symbol] represents the A-R^{a} bond.

In some embodiments, A is a five-membered ring. In some instances, A is a five-membered heterocyclic ring. In some instances, A is a five-membered heteroaryl ring. In some embodiments, A is a five-membered ring and the compound is of formula (IVB):

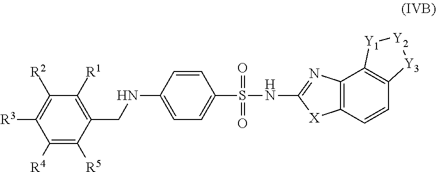

where Y₁, Y₂, Y₃ and Y₄ are each independently C, N or O.

In some embodiments, Y₁, Y₂ and Y₃ are each C. In some embodiments, Y₂ is C and Y₁, and Y₃ are each O. In some embodiments, Y₁ is N; and Y₂ and Y₃ are C. In some embodiments, Y₃ is N; and Y₁ and Y₂ are C. In some embodiments, Y₁ is O; Y₂ is C and Y₃ is N. In some embodiments, Y₁ is N; Y₂ is C and Y₃ is O. In some embodiments, A is unsubstituted. In some embodiments, A is monosubstituted. In other embodiments, A is di-substituted. In other embodiments, A is tri-substituted. In some instances, n is 1 or 2 and each R^{a} is independently selected from:

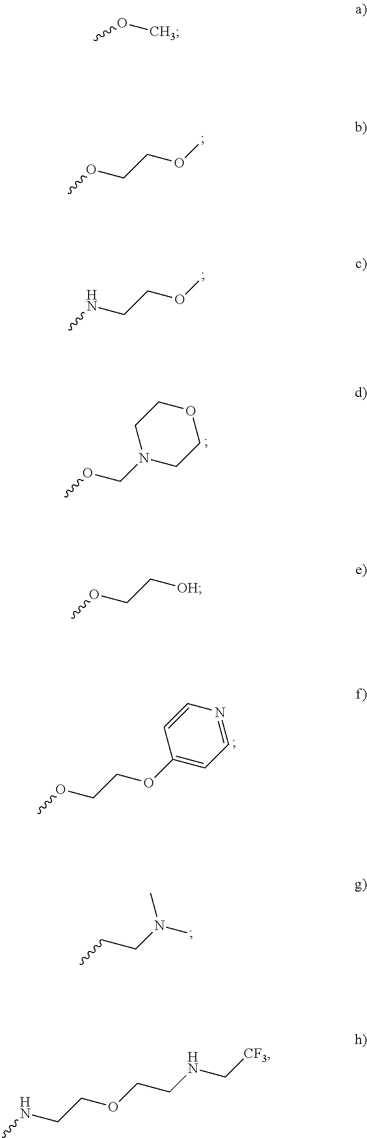

where [symbol] represents the A-R^{a} bond.

In certain embodiments, the compound is 4-((2-hydroxy-3-methoxybenzyl)amino)-N-(naphtho[1,2-d]thiazol-2-yl)benzenesulfonamide:

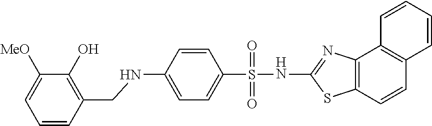 [SMILES: COc1cccc(CNc2ccc(S(=O)(=O)Nc3nc4c(ccc5ccccc54)s3)cc2)c1O]

or a pharmaceutically acceptable salt, solvate or hydrate thereof.

Aspects of the disclosure also include methods for modulating or inhibiting a lipoxygenase enzyme (e.g., 12/15-lipoxygenase) by contacting a cell with an amount of the subject compositions. In some instances, the cell is contacted with the composition in vitro. In other instances, the cell is contacted with the composition in vivo. In some instances, methods include contacting one or more of the compositions described herein with cells having a lipoxygenase enzyme (e.g., human reticulocyte 15-lipoxygenase-2 (h15-LOX-1)) in a manner sufficient to modulate the generation of hydroperoxy eicosatetraeneoic acids (HpETEs) in cells (e.g., human cells). In some embodiments, methods include modulating ferroptosis. In some embodiments, methods include modulating ferroptosis in a manner sufficient to reduce the accumulation of hydroperoxy membrane phospholipids in the contacted cells. In some embodiments, methods include modulating eicosanoid mediator biosynthesis from leukotrienes (LTs) to pro-resolving mediator class of lipoxins (LXs). In some embodiments, methods include modulating h15-LOX-1 in a manner sufficient to reduce foam cell formation and atherosclerotic plaque accumulation. In some embodiments, methods include modulating h15-LOX-1 in a manner sufficient to reduce foam cell formation or atherosclerotic plaque accumulation to treat stroke in a subject.

In some embodiments, methods include treating or preventing a lipoxygenase mediated disease. In some embodiments, methods include treating or preventing atherosclerotic plaque formation or accumulation. In some embodiments, methods include treating or preventing a cardiovascular disease. In some embodiments, methods include treating or preventing cystic fibrosis lung disease. In some embodiments, methods include treating or preventing a neurodegenerative disease, such as Alzheimer's disease, Parkinson's disease, and Huntington's disease. In some embodiments, methods include administering one or more of the compounds described herein to a subject diagnosed with one or more a cardiovascular disease, cystic fibrosis lung disease and a neurodegenerative disease such as Alzheimer's disease, Parkinson's disease, and Huntington's disease. In certain embodiments, methods include treating a subject that has or is diagnosed as having a disease or disorder selected from: stroke, diabetes, obesity, asthma, glomerulonephritis, osteoporosis, periventricular leukomalacia, cardiac arrest with resuscitation, atherosclerosis, neurodegenerative or neuroinflammatory disorders selected from Parkinson's disease, Alzheimer's disease, Huntington's disease and dementia, cancer, brain injury, a disease involving hypoxia or anoxia, myocardial infarction, cardiovascular disease, chronic heart failure, congestive heart, ischemia selected from cerebral ischemia, retinal ischemia, myocardial ischemia, or post-surgical cognitive dysfunction, inflammatory disease, arterial inflammation, inflammatory bowel disease, Crohn's disease, renal disease, asthma, allergic rhinitis, gout, cardiopulmonary inflammation, rheumatoid arthritis, osteoarthritis, muscle fatigue, acne, dermatitis, or psoriasis), chronic bronchitis, mucus hypersecretion, chronic obstructive pulmonary disease (COPD), pulmonary fibrosis, fibrosis caused by chemotherapy, idiopathic pulmonary fibrosis, cystic fibrosis, adult respiratory distress syndrome, CNS disorders, psychiatric disorders, anxiety, depression, peripheral neuropathy, spinal cord injury, head injury, surgical trauma, allograft tissue or organ transplant rejection, autoimmune disorder, eczema and disorders involving bone loss or bone formation. In certain embodiments, methods include treating a subject for stroke.

### Definitions

The following terms have the following meaning unless otherwise indicated. Any undefined terms have their art recognized meanings.

As used herein, the term “alkyl” by itself or as part of another substituent refers to a saturated branched or straight-chain monovalent hydrocarbon radical derived by the removal of one hydrogen atom from a single carbon atom of a parent alkane. Typical alkyl groups include, but are not limited to, methyl; ethyl, propyls such as propan-1-yl or propan-2-yl; and butyls such as butan-1-yl, butan-2-yl, 2-methyl-propan-1-yl or 2-methyl-propan-2-yl. In some embodiments, an alkyl group comprises from 1 to 20 carbon atoms. In other embodiments, an alkyl group comprises from 1 to 10 carbon atoms. In still other embodiments, an alkyl group comprises from 1 to 6 carbon atoms, such as from 1 to 4 carbon atoms.

“Alkanyl” by itself or as part of another substituent refers to a saturated branched, straight-chain or cyclic alkyl radical derived by the removal of one hydrogen atom from a single carbon atom of an alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkylene” refers to a branched or unbranched saturated hydrocarbon chain, usually having from 1 to 40 carbon atoms, more usually 1 to 10 carbon atoms and even more usually 1 to 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

“Alkenyl” by itself or as part of another substituent refers to an unsaturated branched, straight-chain or cyclic alkyl radical having at least one carbon-carbon double bond derived by the removal of one hydrogen atom from a single carbon atom of an alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” by itself or as part of another substituent refers to an unsaturated branched, straight-chain or cyclic alkyl radical having at least one carbon-carbon triple bond derived by the removal of one hydrogen atom from a single carbon atom of an alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Acyl” by itself or as part of another substituent refers to a radical —C(O)R³⁰, where R³⁰ is hydrogen, alkyl, cycloalkyl, cycloheteroalkyl, aryl, arylalkyl, heteroalkyl, heteroaryl, heteroarylalkyl as defined herein and substituted versions thereof. Representative examples include, but are not limited to formyl, acetyl, cyclohexylcarbonyl, cyclohexylmethylcarbonyl, benzoyl, benzylcarbonyl, piperonyl, succinyl, and malonyl, and the like.

The term “aminoacyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Alkoxy” by itself or as part of another substituent refers to a radical —OR³¹ where R³¹ represents an alkyl or cycloalkyl group as defined herein. Representative examples include, but are not limited to, methoxy, ethoxy, propoxy, butoxy, cyclohexyloxy and the like.

“Alkoxycarbonyl” by itself or as part of another substituent refers to a radical —C(O)OR³¹ where R³¹ represents an alkyl or cycloalkyl group as defined herein. Representative examples include, but are not limited to, methoxycarbonyl, ethoxycarbonyl, propoxycarbonyl, butoxycarbonyl, cyclohexyloxycarbonyl and the like.

“Aryl” by itself or as part of another substituent refers to a monovalent aromatic hydrocarbon radical derived by the removal of one hydrogen atom from a single carbon atom of an aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In certain embodiments, an aryl group comprises from 6 to 20 carbon atoms. In certain embodiments, an aryl group comprises from 6 to 12 carbon atoms. Examples of an aryl group are phenyl and naphthyl.

“Arylalkyl” by itself or as part of another substituent refers to an acyclic alkyl radical in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In certain embodiments, an arylalkyl group is (C₇-C₃₀) arylalkyl, e.g., the alkanyl, alkenyl or alkynyl moiety of the arylalkyl group is (C₁-C₁₀) and the aryl moiety is (C₆-C₂₀). In certain embodiments, an arylalkyl group is (C₇-C₂₀) arylalkyl, e.g., the alkanyl, alkenyl or alkynyl moiety of the arylalkyl group is (C₁-C₈) and the aryl moiety is (C₆-C₁₂).

“Arylaryl” by itself or as part of another substituent, refers to a monovalent hydrocarbon group derived by the removal of one hydrogen atom from a single carbon atom of a ring system in which two or more identical or non-identical aromatic ring systems are joined directly together by a single bond, where the number of such direct ring junctions is one less than the number of aromatic ring systems involved. Typical arylaryl groups include, but are not limited to, biphenyl, triphenyl, phenyl-napthyl, binaphthyl, biphenyl-napthyl, and the like. When the numbers of carbon atoms in an arylaryl group are specified, the numbers refer to the carbon atoms comprising each aromatic ring. For example, (C₅-C₁₄) arylaryl is an arylaryl group in which each aromatic ring comprises from 5 to 14 carbons, e.g., biphenyl, triphenyl, binaphthyl, phenylnapthyl, etc. In certain embodiments, each aromatic ring system of an arylaryl group is independently a (C₅-C₁₄) aromatic. In certain embodiments, each aromatic ring system of an arylaryl group is independently a (C₅-C₁₀) aromatic. In certain embodiments, each aromatic ring system is identical, e.g., biphenyl, triphenyl, binaphthyl, trinaphthyl, etc.

“Cycloalkyl” by itself or as part of another substituent refers to a saturated or unsaturated cyclic alkyl radical. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane and the like. In certain embodiments, the cycloalkyl group is (C₃-C₁₀) cycloalkyl. In certain embodiments, the cycloalkyl group is (C₃-C₇) cycloalkyl.

“Cycloheteroalkyl” or “heterocyclyl” by itself or as part of another substituent, refers to a saturated or unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidine, quinuclidine and the like.

“Heteroalkyl, Heteroalkanyl, Heteroalkenyl and Heteroalkynyl” by themselves or as part of another substituent refer to alkyl, alkanyl, alkenyl and alkynyl groups, respectively, in which one or more of the carbon atoms (and any associated hydrogen atoms) are independently replaced with the same or different heteroatomic groups. Typical heteroatomic groups which can be included in these groups include, but are not limited to, —O—, —S—, —S—S—, —O—S—, —NR³⁷R³⁸—, ═N—N═, —N═N—, —N═N—NR³⁹R⁴⁰, —PR⁴¹—, —P(O)₂—, —POR⁴²—, —O—P(O)₂—, —S—O—, —S—(O)—, —SO₂—, —SnR⁴³R⁴⁴— and the like, where R³⁷, R³⁸, R³⁹, R⁴⁰, R⁴¹, R⁴², R⁴³ and R⁴⁴ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl” by itself or as part of another substituent, refers to a monovalent heteroaromatic radical derived by the removal of one hydrogen atom from a single atom of a heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, benzodioxole and the like. In certain embodiments, the heteroaryl group is from 5-20 membered heteroaryl. In certain embodiments, the heteroaryl group is from 5-10 membered heteroaryl. In certain embodiments, heteroaryl groups are those derived from thiophene, pyrrole, benzothiophene, benzofuran, indole, pyridine, quinoline, imidazole, oxazole and pyrazine.

“Heteroarylalkyl” by itself or as part of another substituent, refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heterorylalkynyl is used. In certain embodiments, the heteroarylalkyl group is a 6-30 membered heteroarylalkyl, e.g., the alkanyl, alkenyl or alkynyl moiety of the heteroarylalkyl is 1-10 membered and the heteroaryl moiety is a 5-20-membered heteroaryl. In certain embodiments, the heteroarylalkyl group is 6-20 membered heteroarylalkyl, e.g., the alkanyl, alkenyl or alkynyl moiety of the heteroarylalkyl is 1-8 membered and the heteroaryl moiety is a 5-12-membered heteroaryl.

“Aromatic Ring System” by itself or as part of another substituent, refers to an unsaturated cyclic or polycyclic ring system having a conjugated z electron system. Specifically included within the definition of “aromatic ring system” are fused ring systems in which one or more of the rings are aromatic and one or more of the rings are saturated or unsaturated, such as, for example, fluorene, indane, indene, phenalene, etc. Typical aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Heteroaromatic Ring System” by itself or as part of another substituent, refers to an aromatic ring system in which one or more carbon atoms (and any associated hydrogen atoms) are independently replaced with the same or different heteroatom. Typical heteroatoms to replace the carbon atoms include, but are not limited to, N, P, O, S, Si, etc. Specifically included within the definition of “heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Typical heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Substituted” refers to a group in which one or more hydrogen atoms are independently replaced with the same or different substituent(s). Typical substituents include, but are not limited to, alkylenedioxy (such as methylenedioxy), -M, —R⁶⁰, —O—, ═O, —OR⁶⁰, —SR⁶⁰, —S—, ═S, —NR⁶⁰R⁶¹═NR⁶⁰—CF₃, —CN, —OCN, —SCN, —NO, —NO₂, ═N₂, —N₃, —S(O)₂O—, —S(O)₂OH, —S(O)₂R⁶⁰, —OS(O)₂O—, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O—, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹ and —C(NR⁶²)NR⁶⁰R⁶¹ where M is halogen; R⁶⁰, R⁶¹, R⁶² and R⁶³ are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R⁶⁰ and R⁶¹ together with the nitrogen atom to which they are bonded form a cycloheteroalkyl or substituted cycloheteroalkyl ring; and R⁶⁴ and R⁶⁵ are independently hydrogen, alkyl, substituted alkyl, aryl, cycloalkyl, substituted cycloalkyl, cycloheteroalkyl, substituted cycloheteroalkyl, aryl, substituted aryl, heteroaryl or substituted heteroaryl, or optionally R⁶⁴ and R⁶⁵ together with the nitrogen atom to which they are bonded form a cycloheteroalkyl or substituted cycloheteroalkyl ring. In certain embodiments, substituents include -M, —R⁶⁰, ═O, —OR⁶⁰, —SR⁶⁰, —S, ═S, —NR⁶⁰R⁶¹, ═NR⁶, —CF₃, —CN, —OCN, —SCN, —NO, —NO₂, ═N₂, —N₃, —S(O)₂R⁶⁰, —OS(O)₂O—, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O—, —NR⁶²C(O)NR⁶⁰R⁶¹. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —P(O)(OR⁶⁰)(O⁻), —OP(O)(OR⁶⁰)(OR⁶¹), —C(O)R⁶⁰, —C(O)OR⁶⁰, —C(O)NR⁶⁰R⁶¹, —C(O)O. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —OP(O)(OR⁶⁰)(OR⁶¹), —C(O)R⁶⁰, —C(O)OR⁶⁰, —C(O)O—, where R⁶⁰, R⁶¹ and R⁶² are as defined above. For example, a substituted group may bear a methylenedioxy substituent or one, two, or three substituents selected from a halogen atom, a (1-4C)alkyl group and a (1-4C)alkoxy group.

“Pharmaceutically acceptable carrier” refers to a diluent, adjuvant, excipient or vehicle with, or in which a compound is administered.

### DETAILED DESCRIPTION

Compositions for inhibiting a lipoxygenase enzyme (e.g., human reticulocyte 12/15-lipoxygenase (12/15-LOX) are provided. Compositions according to certain embodiments include a first solvent, a second solvent and a lipoxygenase inhibitor. In certain instances, the lipoxygenase inhibitor is present in the composition (e.g., compositions formulated for intravenous administration) at a concentration of 30 mg/mL or greater. Methods for treating or preventing a lipoxygenase (e.g., human epithelial 12/15-lipoxygenase (12/15-LOX))-mediated disease or disorder are also provided.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the compounds and methods have or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the chemical groups represented by the variables are specifically embraced by the present invention and are disclosed herein just as if each and every combination was individually and explicitly disclosed, to the extent that such combinations embrace compounds that are stable compounds (i.e., compounds that can be isolated, characterised, and tested for biological activity). In addition, all sub-combinations of the chemical groups listed in the embodiments describing such variables are also specifically embraced by the present invention and are disclosed herein just as if each and every such sub-combination of chemical groups was individually and explicitly disclosed herein.

Reference will now be made in detail to various embodiments. It will be understood that the invention is not limited to these embodiments. To the contrary, it is intended to cover alternatives, modifications, and equivalents as may be included within the spirit and scope of the allowed claims.

### Compositions for Modulating A Lipoxygenase Enzyme

In embodiments, compositions include a solvent component having a first solvent and a second solvent and a lipoxygenase inhibitor present in the composition in a concentration of 30 mg/mL or more. In certain instances, the composition is formulated for intravenous administration to a subject. In some cases, the intravenous composition is formulated for bolus injection. In other cases, the intravenous composition is formulated for sustained infusion, such as over a period of 1 minute or more, such as 5 minutes or more, such as 10 minutes or more, such as 15 minutes or more, such as 30 minutes or more, such as 60 minutes or more, such as 2 hours or more, such as 4 hours or more and including by infusion over a period of 8 hours or more.

In some embodiments, the first solvent includes a polar solvent. In some instances, the polar solvent is chosen from dimethylacetamide (DMA) or diethylacetamide (DEA), chloroform, dichloromethane, tetrahydrofuran, dichloroethane, acetone, dioxane, ethyl acetate, dimethylsulfoxide, aniline, diethylamine, nitromethane, acetonitrile, pyridine, isopropanol, ethanol, methanol, ethylene glycol, acetic acid, water and combinations thereof. In some instances, the first solvent includes dimethylacetamide (DMA) or diethylacetamide (DEA) or a combination thereof.

In certain embodiments, the first solvent of the solvent component includes a non-ionic surfactant, such as a lipid-based formulation that solubilizes or increases bio-availability of the lipoxygenase inhibitors described herein. In some instances, the first solvent includes a polyoxyethylene ester of 12-hydroxysteraric acid, a polyethoxylated castor oil, a polyvinyl caprolactam-polyvinyl acetate-polyethylene glycol, a propylene glycol caprylate, a diethylene glycol monomethyl ether, a caprylocaproyl macrogolglyceride, a polyoxyl-6 glyceride and a propylene glycol monolaurate. In certain instances, the polyoxyethylene ester of 12-hydroxysteraric acid includes a solvent that is or is substantially the same as Solutol. In certain instances, the polyethoxylated castor oil includes a solvent that is or is substantially the same as Kolliphor. In certain instances, the solvent includes a solvent that is or is substantially the same as Kolliphor EL. In certain instances, the solvent includes a solvent that is or is substantially the same as Kolliphor RH40. In certain instances, the solvent includes a solvent that is or is substantially the same as Kolliphor P188 Geismar. In certain instances, the polyvinyl caprolactam-polyvinyl acetate-polyethylene glycol includes a solvent that is or is substantially the same as Soluplus. In certain instances, the propylene glycol caprylate includes a solvent that is or is substantially the same as Capryol. In certain instances, the diethylene glycol monomethyl ether includes a solvent that is or is substantially the same as Transcutol. In certain instances, the caprylocaproyl macrogolglyceride includes a solvent that is or is substantially the same as Labrasol. In certain instances, the polyoxyl-6 glyceride includes a solvent that is or is substantially the same as Labrafil. In certain instances, the propylene glycol monolaurate includes a solvent that is or is substantially the same as Lauroglycol.

In some embodiments, the second solvent of the solvent component of the lipoxygenase compositions include a polyethyelene glycol, such as PEG400. In some embodiments, the second solvent of the solvent component of the lipoxygenase compositions include an alcohol solvent, such as methanol, ethanol, propanol, isopropanol, butanol, isobutanol or pentanol. In certain embodiments, the second solvent includes a high boiling point polar solvent, such as a glycol ether. In certain cases, the at least one high boiling point solvent is chosen from a glycol ether, a dialkyl ether and an alkyl ether ester. In certain cases, the at least one high boiling point solvent is chosen from ethylene glycol monomethyl ether, ethylene glycol monoethyl ether, ethylene glycol monopropyl ether, ethylene glycol monoisopropyl ether, ethylene glycol monobutyl ether, ethylene glycol monophenyl ether, ethylene glycol monobenzyl ether, propylene glycol methyl ether, diethylene glycol monomethyl ether, diethylene glycol monomethyl ether, diethylene glycol monoethyl ether, diethylene glycol mono-n-butyl ether, dipropyleneglycol methyl ether, ethylene glycol dimethyl ether, ethylene glycol diethyl ether, ethylene glycol dibutyl ether, ethylene glycol methyl ether acetate, ethylene glycol monoethyl ether acetate, ethylene glycol monobutyl ether acetate and propylene glycol methyl ether acetate.

In certain embodiments, the solvent component of the subject compositions includes dimethylacetamide (DMA) and polyethylene glycol 400 (PEG400). In certain embodiments, the solvent component of the subject compositions includes diethylacetamide (DEA) and polyethyelene glycol 400. In certain embodiments, the solvent component of the subject compositions includes solutol and PEG400. In certain embodiments, the solvent component of the subject compositions includes DMA, ethanol and PEG400. In certain embodiments, the solvent component of the subject compositions includes dimethyl sulfoxide (DMSO) and PEG400. In certain embodiments, the solvent component of the subject compositions includes Kolliphor EL and PEG400. In certain embodiments, the solvent component of the subject compositions includes Kolliphor EL and ethanol. In certain embodiments, the solvent component of the subject compositions includes Kolliphor RH40 and PEG400. In certain embodiments, the solvent component of the subject compositions includes Kolliphor P188 Geismar and PEG400. In certain embodiments, the solvent component of the subject compositions includes Soluplus and PEG400. In certain embodiments, the solvent component of the subject compositions includes Capryol and PEG400. In certain embodiments, the solvent component of the subject compositions includes Transcutol and PEG400. In certain embodiments, the solvent component of the subject compositions includes Labrafil and PEG400.

In certain embodiments, the solvent component of the subject compositions includes Labrasol and PEG400. In certain embodiments, the solvent component of the subject compositions includes lauroglycol and PEG400.

The amount of first solvent and second solvent in the composition may vary, such as where 0.01 equivalents of the first solvent is combined with the second solvent, such as 0.1 equivalents, such as 0.2 equivalents or more, such as 0.3 equivalents or more, such as 0.4 equivalents or more, such as 0.5 equivalents or more, such as 0.6 equivalents or more, such as 0.7 equivalents or more, such as 0.8 equivalents or more, such as 0.9 equivalents or more, such as 1.0 equivalents or more, such as 1.1 equivalents or more, such as 1.2 equivalents or more, such as 1.3 equivalents or more, such as 1.4 equivalents or more, such as 1.5 equivalents or more, such as 1.6 equivalents or more, such as 1.7 equivalents or more, such as 1.8 equivalents or more, such as 1.9 equivalents or more, such as 2 equivalents or more, and including 3 equivalents or more.

The amount of first solvent and second solvent present in the composition may range, such as in a weight ratio of from 1:99 to 99:1, such as from 2:98 to 98:2, such as from 3:97 to 97:3, such as from 4:96 to 96:4, such as from 5:95 to 95:5, such as from 6:94 to 94:6, such as from 7:93 to 93:7, such as from 8:92 to 92:8, such as from 9:91 to 91:9, such as from 10:90 to 90:10, such as from 20:80 to 80:20, such as from 30:70 to 70:30, such as from 40:60 to 60:40 and including a weight ratio of the first solvent to second solvent of 50:50. In certain instances, the volume ratio of first solvent to second solvent ranges from 1:99 to 99:1, such as from 2:98 to 98:2, such as from 3:97 to 97:3, such as from 4:96 to 96:4, such as from 5:95 to 95:5, such as from 6:94 to 94:6, such as from 7:93 to 93:7, such as from 8:92 to 92:8, such as from 9:91 to 91:9, such as from 10:90 to 90:10, such as from 20:80 to 80:20, such as from 30:70 to 70:30, such as from 40:60 to 60:40 and including a volume ratio of the first solvent to second solvent of 50:50. In certain instances, the molar ratio of first solvent to second solvent ranges from 1:99 to 99:1, such as from 2:98 to 98:2, such as from 3:97 to 97:3, such as from 4:96 to 96:4, such as from 5:95 to 95:5, such as from 6:94 to 94:6, such as from 7:93 to 93:7, such as from 8:92 to 92:8, such as from 9:91 to 91:9, such as from 10:90 to 90:10, such as from 20:80 to 80:20, such as from 30:70 to 70:30, such as from 40:60 to 60:40 and including a molar ratio of the first solvent to second solvent of 50:50.

In some embodiments, the composition is formulated for intravenous administration to a subject, such as by bolus injection or by sustained infusion. In certain instances, the composition is formulated such that the concentration of the lipoxygenase inhibitor present in the composition is 25 mg/mL or more, such as 30 mg/mL or more, such as 35 mg/mL or more, such as 40 mg/mL or more, such as 45 mg/mL or more, such as 50 mg/mL or more, such as 55 mg/mL or more, such as 60 mg/mL or more, such as 65 mg/mL or more, such as 70 mg/mL or more, such as 75 mg/mL or more, such as 80 mg/mL or more, such as 85 mg/mL or more, such as 90 mg/mL or more, such as 95 mg/mL or more, such as 100 mg/mL or more, such as 110 mg/mL or more, such as 120 mg/mL or more, such as 130 mg/mL or more, such as 140 mg/mL or more, such as 150 mg/mL or more, such as 175 mg/mL or more, such as 200 mg/mL or more and including 250 mg/mL or more.

In some embodiments, the lipoxygenase inhibitor present in compositions of interest is a compound of Formula (I):

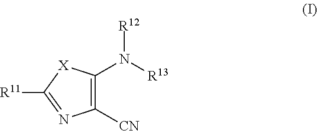

or a pharmaceutically acceptable salt thereof, in which.
X is O or S;
R¹¹ is an aryl, heteroaryl, cyclyl, or heterocyclyl, each of which can be optionally substituted; and
R¹² and R¹³ are independently hydrogen, halogen, alkyl, alkenyl, alkynyl, aralkyl, acyl, aryl, heteroaryl, cyclyl or heterocyclyl, each of which can be optionally substituted
or a pharmaceutically acceptable salt thereof.

In some embodiments, R¹¹ is an aryl or heteroaryl, each of which can be optionally substituted. In some embodiments, R¹¹ is 1-naphthyl, 2-naphthyl, 6-isoquinolinyl, 2,3-dichlorophenyl, or 3,4-dichlorophenyl. In some embodiments, R¹¹ is a monocyclic aryl, bicyclic aryl, monocyclic heteroaryl, or bicyclic heteroaryl, each of which can be optionally substituted.

In some embodiments, R¹¹ is an optionally substituted phenyl. When the phenyl group is substituted, the substituent can be present at the ortho, meta, or para position on the phenyl relative to the rest of the compound. In some embodiments, the optionally substituted aryl or heteroaryl is substituted with one or more substituents selected from amino, halogen, hydroxyl, thiol, methoxy, methylthioxy, carboxyl, nitro, cyano, and any combinations thereof. In some embodiments, the heteroaryl comprises one or more heteroatoms (e.g., 1, 2, 3, 4, 5, or more) selected from O, S, N, and a combination thereof. In some embodiments, R¹² is hydrogen, alkyl, aralkyl, acyl, aryl, or heterocyclyl, each of which can be optionally substituted. In some embodiments, R¹² is hydrogen, methyl, ethyl, propyl, butyl, pentyl, or phenyl. In some embodiments, R¹³ is hydrogen, alkyl, aralkyl, acyl, aryl, or heterocyclyl, each of which can be optionally substituted. In some embodiments, R¹² and R¹³ are independently hydrogen, alkyl, aralkyl, alkenyl, acyl, aryl, or heterocyclyl, each of which can be optionally substituted. In some embodiments, the alkyl is C₁-C₁₀ alkyl. In some embodiments, the alkyl is C₁-C₆ alkyl such as methyl, ethyl, propyl, isopropyl, butyl, sec-butyl, isobutyl, tert-butyl, pentyl, neopentyl, and hexyl. In some embodiments, R¹³ is hydrogen, methyl, ethyl, propyl, butyl, pentyl, or phenyl. In some embodiments, R¹² is hydrogen and R¹³ is hydrogen, methyl, ethyl, propyl, butyl, pentyl, or phenyl. In some embodiments, R¹² and R¹³ are both methyl. In some embodiments, X is O. In some embodiments, X is S.

In embodiments, “salts” of the compounds of the present disclosure may include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

The term “solvate” as used herein refers to a complex or aggregate formed by one or more molecules of a solute, e.g. a compound of Formula (I) or a salt thereof, and one or more molecules of a solvent. Such solvates may be crystalline solids having a substantially fixed molar ratio of solute and solvent. Representative solvents include by way of example, water, methanol, ethanol, isopropanol, acetic acid, and the like. When the solvent is water, the solvate formed is a hydrate.

In some embodiments, the lipoxygenase inhibitor present in compositions of interest is a compound of Formula (Ia):

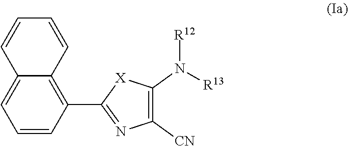

or a pharmaceutically acceptable salt thereof, in which:
X is O or S;
R¹¹ and R¹³ are independently hydrogen, halogen, alkyl, alkenyl, alkynyl, aralkyl, acyl, aryl, heteroaryl, cyclyl or heterocyclyl, each of which can be optionally substituted
or a pharmaceutically acceptable salt thereof.

In some embodiments, R¹² is hydrogen, alkyl, aralkyl, acyl, aryl, or heterocyclyl, each of which can be optionally substituted. In some embodiments, R¹² is hydrogen, methyl, ethyl, propyl, butyl, pentyl, or phenyl. In some embodiments, R¹³ is hydrogen, alkyl, aralkyl, acyl, aryl, or heterocyclyl, each of which can be optionally substituted. In some embodiments, R¹² and R¹³ are independently hydrogen, alkyl, aralkyl, alkenyl, acyl, aryl, or heterocyclyl, each of which can be optionally substituted. In some embodiments, the alkyl is C₁-C₁₀ alkyl. In some embodiments, the alkyl is C₁-C₆ alkyl such as methyl, ethyl, propyl, isopropyl, butyl, sec-butyl, isobutyl, tert-butyl, pentyl, neopentyl, and hexyl. In some embodiments, R¹³ is hydrogen, methyl, ethyl, propyl, butyl, pentyl, or phenyl. In some embodiments, R¹² is hydrogen and R³ is hydrogen, methyl, ethyl, propyl, butyl, pentyl, or phenyl. In some embodiments, R¹² and R¹³ are both methyl. In some embodiments, X is O. In some embodiments, X is S.

In certain instances, the lipoxygenase inhibitor is selected from 5-(methylamino)-2-naphthalen-1-yl-1,3-oxazole-4-carbonitrile (ML351), 2-(2,3-dichlorophenyl)-5-(methylamino)-1,3-oxazole-4-carbonitrile, 2-(3,4-dichlorophenyl)-5-(methylamino)-1,3-oxazole-4-carbonitrile, 5-(methylamino)-2-naphthalen-1-yl-1,3-thiazole-4-carbonitrile or a pharmaceutically acceptable salt thereof.

In some embodiments, the lipoxygenase inhibitor is selected from:

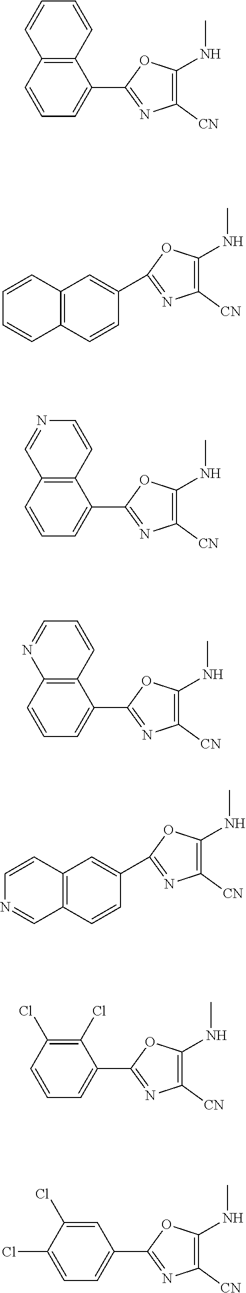

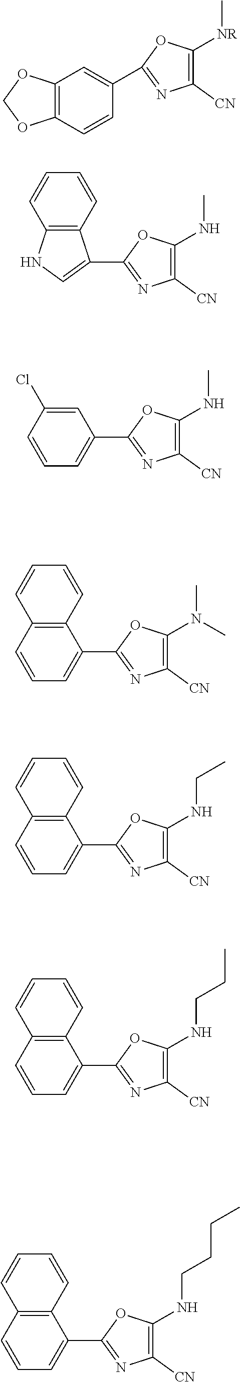

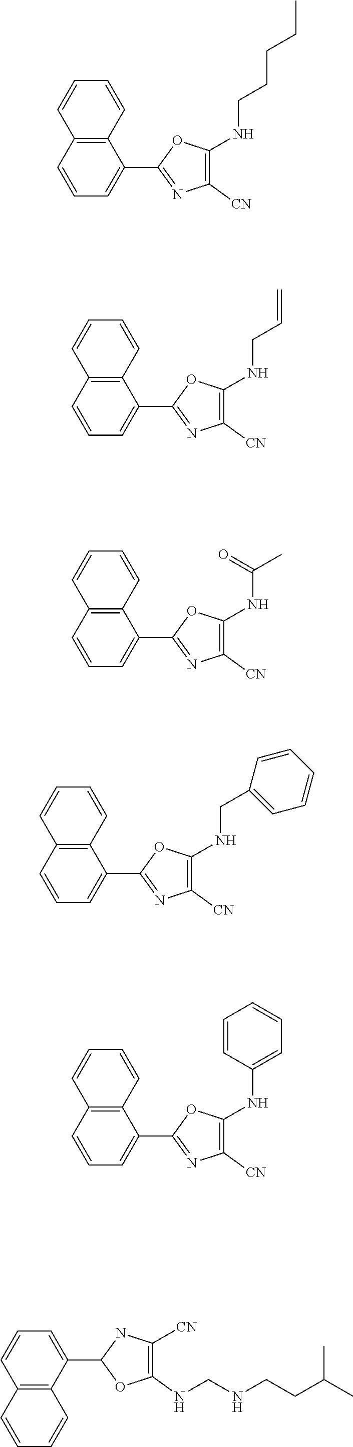

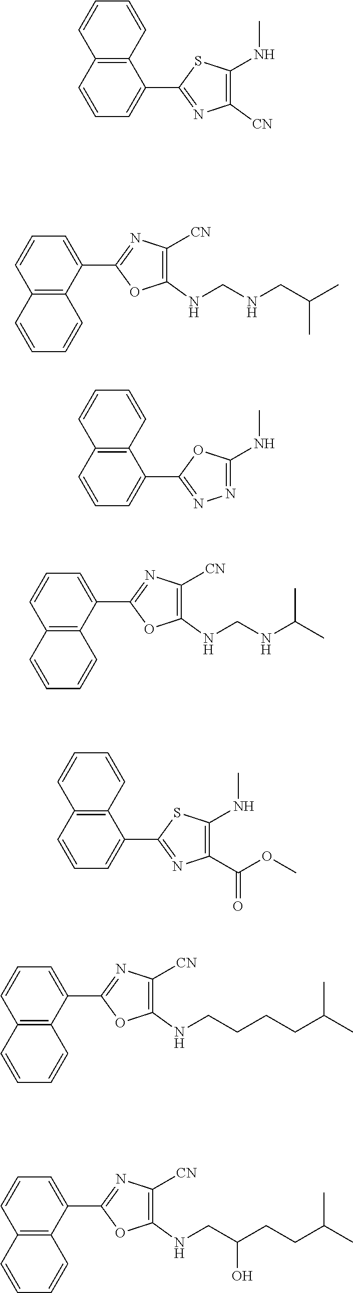

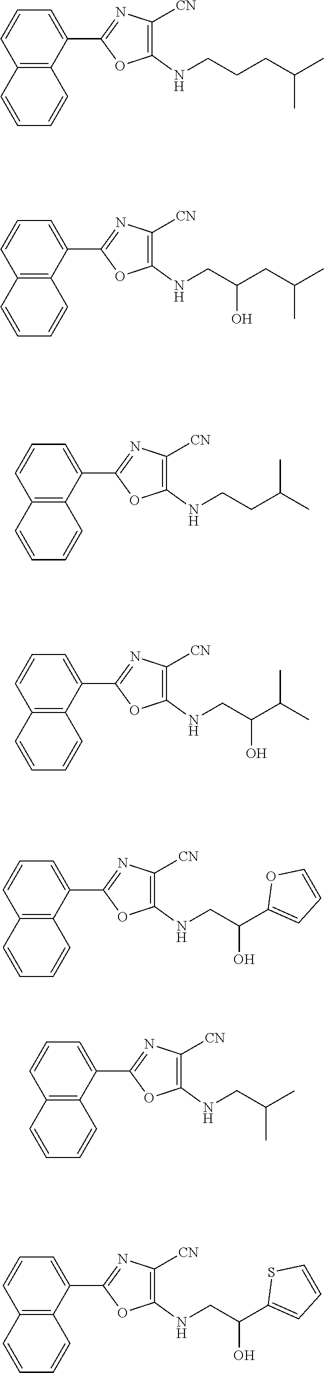

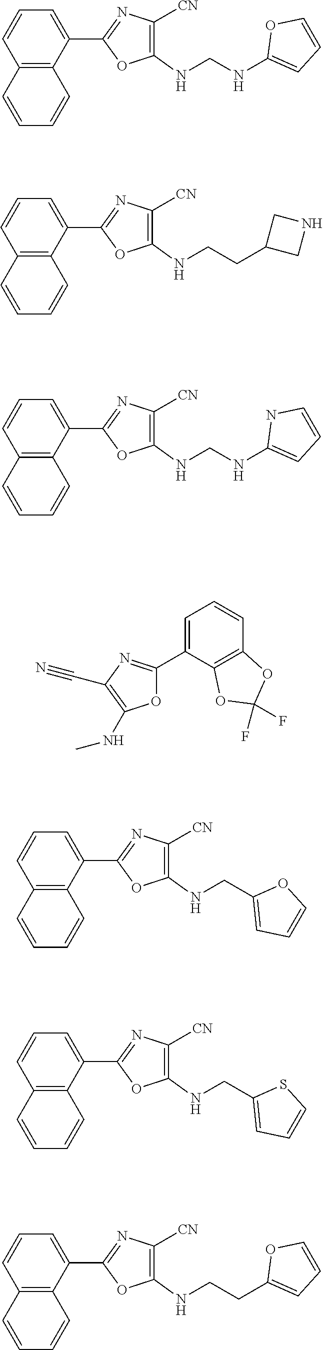

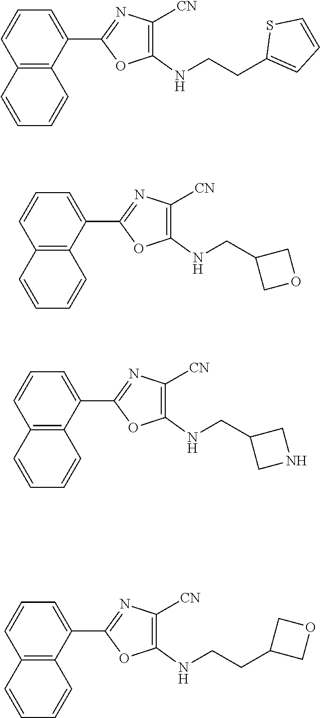 [SMILES: N#Cc1nc(-c2cccc3ccccc23)oc1NCCc1cccs1 ; N#Cc1nc(-c2cccc3ccccc23)oc1NCC1COC1 ; N#Cc1nc(-c2cccc3ccccc23)oc1NCC1CNC1 ; N#Cc1nc(-c2cccc3ccccc23)oc1NCCC1COC1]

In certain embodiments, lipoxygenase inhibitors include compounds of Formula (I) or Formula (Ia), such as described in U.S. Pat. No. 10,287,279, the disclosure of which is herein incorporated by reference.

In some embodiments, the lipoxygenase inhibitor present in compositions of interest is a a compound of Formula (II):

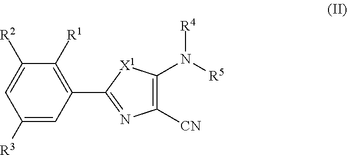

or a pharmaceutically acceptable salt thereof, wherein:
X¹ is selected from O and S;
R¹, R², and R³ are each independently selected from halo, CN, C₁₋₃ alkyl, C₁₋₃ haloalkyl, C₁₋₃ alkoxy, and C₁₋₃ haloalkoxy;
R⁴ is selected from H, C₁₋₃ alkyl, and HO—C₁₋₃ alkylene;
R⁵ is selected from C₁₋₆ alkyl, C₂₋₆ alkenyl, C₂₋₆ alkynyl, C(O)OR^{a1}, C(O)N(R^{a1})₂, P(═O)(OR^{a1})₂, and C(O)R^{b1}; wherein said C₁₋₆ alkyl, C₂₋₆ alkenyl, and C₂₋₆ alkynyl are each optionally substituted with a substituent selected from OR^{a1} and OP(═O)(OR^{a1})₂;
each R^{a1} is independently selected from H, C₁₋₆ alkyl, C₆₋₁₀ aryl, C₁₋₆ alkyl-C₆₋₁₀ aryl, and C₁₋₆ alkyl-C₆₋₁₀ aryl-C₁₋₆ alkyl, wherein said C₁₋₆ alkyl, C₆₋₁₀ aryl, C₁₋₆ alkyl-C₆₋₁₀ aryl, and C₁₋₆ alkyl-C₆₋₁₀ aryl-C₁₋₆ alkyl are each optionally substituted with a substituent selected from amino, C₁₋₆ alkylamino, (C₁₋₆ haloalkyl)amino, di(C₁₋₆ alkyl)amino, (C₁₋₆ alkyl)(C₁₋₆ haloalkyl)amino, (C₆₋₁₀ aryl)amino, (C₆₋₁₀ aryl)(C₁₋₆ alkyl)amino, (5-6-membered heteroaryl)amino, (5-6-membered heteroaryl)(C₁₋₆ alkyl)amino, C₆₋₁₀ aryl, 4-6 membered heterocycloalkyl, 5-6-membered heteroaryl, and OR^{a2}, wherein said C₆₋₁₀ aryl, 4-6 membered heterocycloalkyl, and 5-6-membered heteroaryl are each optionally substituted with 1, 2, or 3 substituents independently selected from amino, C₁₋₆ alkylamino, di(C₁₋₆ alkyl)amino, carboxy, and halo;
each R^{a2} is independently selected from H, C₁₋₃ alkyl, C₁₋₃ haloalkyl, C₁₋₃ alkoxy-C₁₋₃ alkyl, 4-7 membered heterocycloalkyl-C₁₋₃ alkyl, 5-6-membered heteroaryloxy-C₁₋₃ alkyl, C₆₋₁₀ aryl, and 5-6-membered heteroaryl, wherein said C₆₋₁₀ aryl and 5-6-membered heteroaryl are each optionally substituted with 1, 2, or 3 substituents independently selected from halo, C₁₋₃ alkoxy, C₁₋₃ haloalkoxy, C₁₋₃ alkyl, and C₁₋₃ haloalkyl; and
R^{b1} is C₁₋₆ alkyl, optionally substituted with a substituent selected from amino, C₁₋₆ alkylamino, di(C₁₋₆ alkyl)amino, and 4-7 membered heterocycloalkyl ring comprising at least one N atom or a pharmaceutically acceptable salt thereof.

In some embodiments, R¹ is halo. In some embodiments, R¹ is CN. In some embodiments, R¹ is C₁₋₃ alkyl. In some embodiments, R¹ is C₁₋₃ haloalkyl. In some embodiments, R² is halo. In some embodiments, R² is CN. In some embodiments, R² is C₁₋₃ alkyl. In some embodiments, R² is C₁₋₃ haloalkyl. In some embodiments, R³ is halo. In some embodiments, R³ is CN. In some embodiments, R³ is C₁₋₃ alkyl. In some embodiments, R³ is C₁₋₃ haloalkyl. In some embodiments, R¹, R², and R³ are each halo. In some embodiments:

R² and R³ are each halo; and R¹ is selected from C₁₋₃ alkyl, C₁₋₃ alkoxy, and C₁₋₃ haloalkoxy.

In some embodiments: R¹ and R³ are each halo; and R² is selected from C₁₋₃ alkyl, C₁₋₃ alkoxy, and C₁₋₃ haloalkyl. In some embodiments: R¹ and R² are each halo; and R³ is selected from C₁₋₃ alkyl, C₁₋₃ haloalkyl, and CN. In some embodiments, R⁵ is C₁₋₃ alkyl.

In some embodiments, R⁵ is C₂₋₆ alkenyl, optionally substituted with OR^{a1}. In some embodiments, R⁵ is C₂₋₆ alkynyl, optionally substituted with OR^{a1}. In some embodiments, R⁵ is C₁₋₆ alkyl substituted with OP(═O)(OR^{a1})₂. In some embodiments, R^{a1} is H. In some embodiments, R^{a1} is C₁₋₆ alkyl, optionally substituted with C₆₋₁₀ aryl or In some embodiments, R⁴ is H. In some embodiments, R⁵ is C(O)OR^{a1}. In some embodiments, R^{a1} is C₁₋₆ alkyl, optionally substituted with a substituent selected from amino, C₁₋₆ alkylamino, and di(C₁₋₆ alkyl)amino. In some embodiments, R⁵ is C(O)R^{b1}. In some embodiments: R⁴ is selected from H, C₁₋₃ alkyl, and HO—C₁₋₃ alkylene; and R⁵ is selected from C₁₋₆ alkyl, C₂₋₆ alkenyl, and C₂₋₆ alkynyl, each of which is optionally substituted with a substituent selected from OR^{a1} and OP(═O)(OR^{a1})₂; and each R is independently selected from H and C₁₋₆ alkyl, wherein said alkyl is optionally substituted with a substituent selected from C₆₋₁₀ aryl and OR^{a2}. In some embodiments: R⁴ is selected from C(O)OR^{a1} and C(O)R^{b1}; R⁵ is C₁₋₃ alkyl; and R^{a1} is C₁₋₆ alkyl, optionally substituted with a substituent selected from amino, C₁₋₆ alkylamino, and di(C₁₋₆ alkyl)amino.

In some embodiments, the lipoxygenase is a compound selected from:

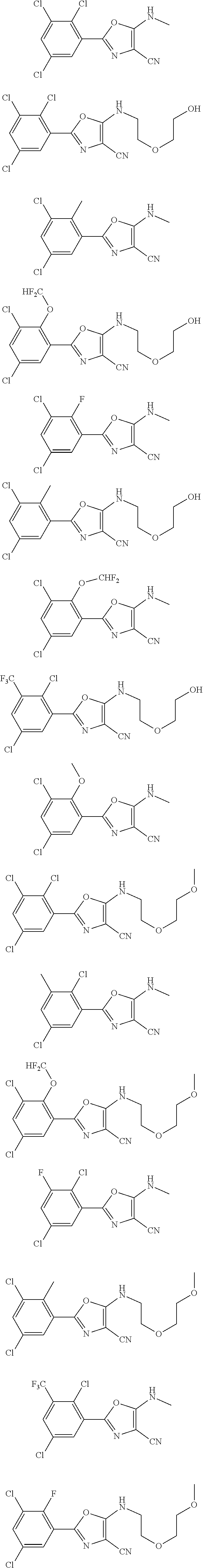

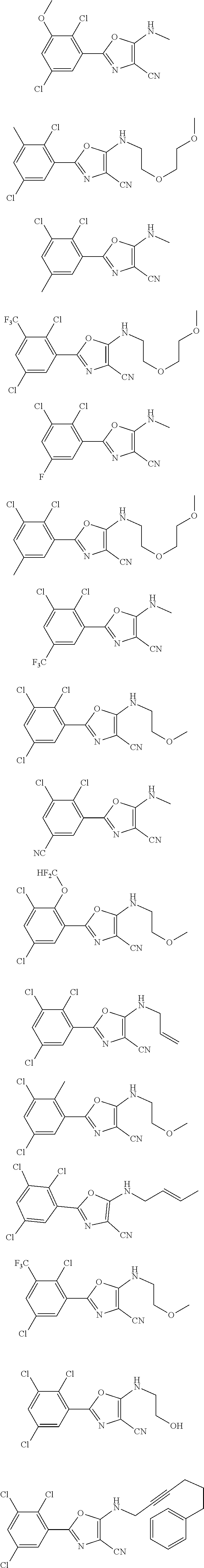

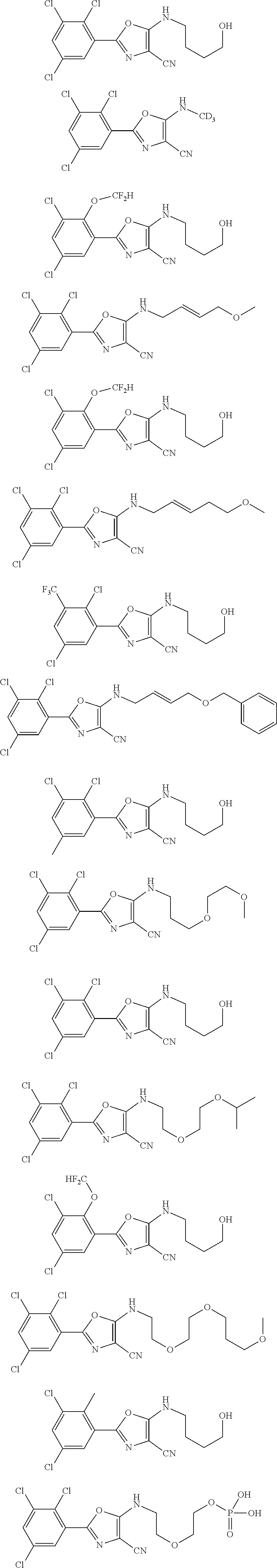

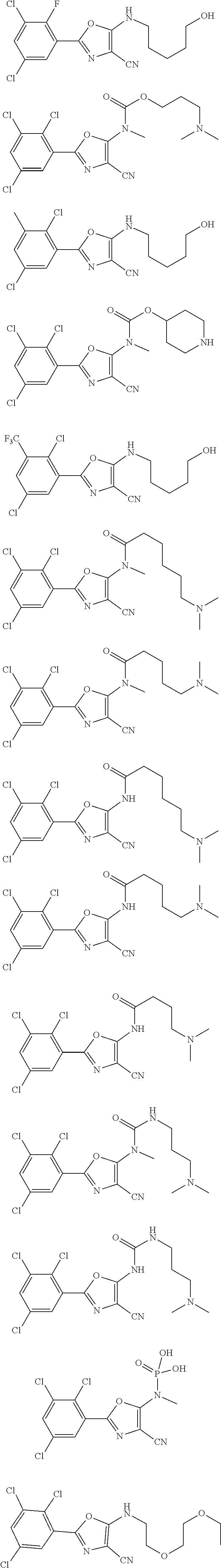

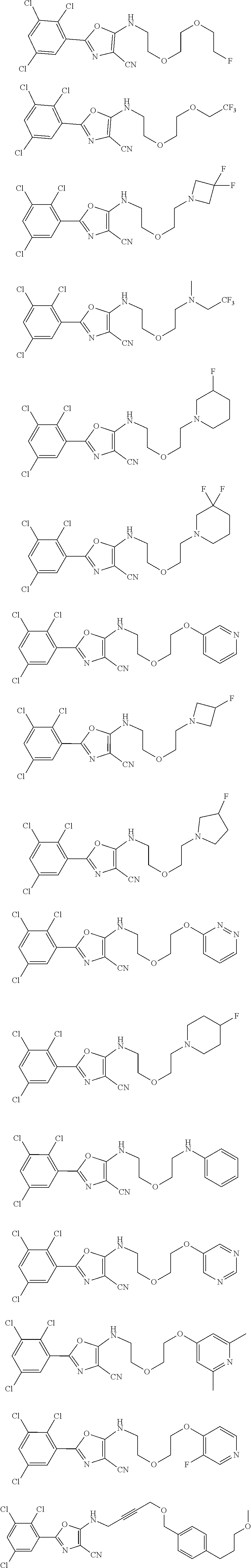

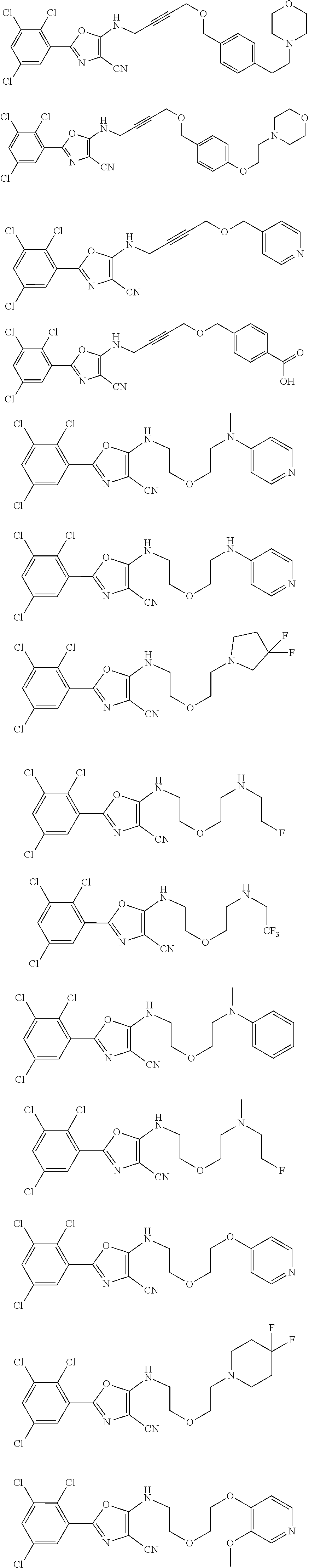

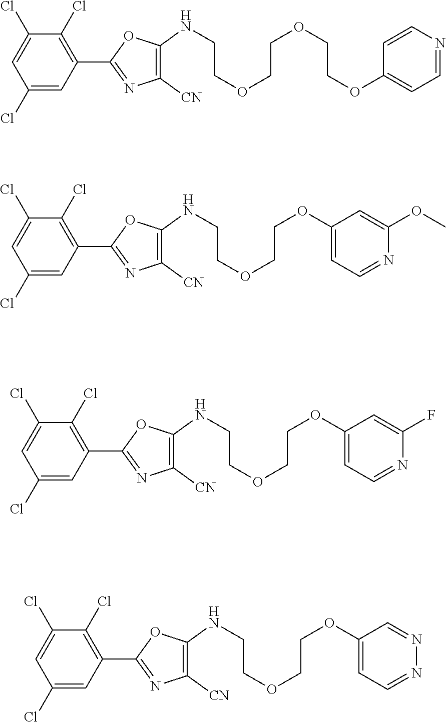 [SMILES: N#Cc1nc(-c2cc(Cl)cc(Cl)c2Cl)oc1NCCOCCOCCOc1ccncc1 ; COc1cc(OCCOCCNc2oc(-c3cc(Cl)cc(Cl)c3Cl)nc2C#N)ccn1 ; N#Cc1nc(-c2cc(Cl)cc(Cl)c2Cl)oc1NCCOCCOc1ccnc(F)c1 ; N#Cc1nc(-c2cc(Cl)cc(Cl)c2Cl)oc1NCCOCCOc1ccnnc1]

or a pharmaceutically acceptable salt thereof.

In certain embodiments, the lipoxygenase inhibitor is a compound selected from:

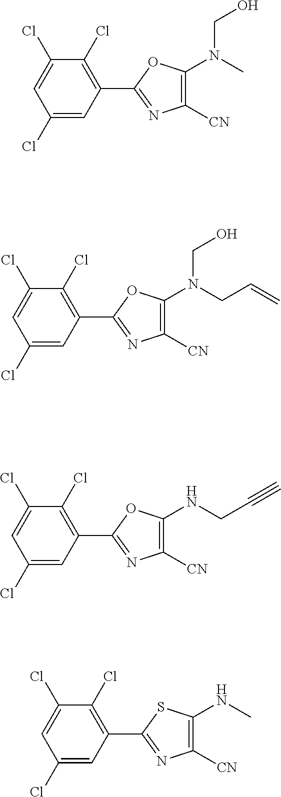 [SMILES: CN(CO)c1oc(-c2cc(Cl)cc(Cl)c2Cl)nc1C#N ; C=CCN(CO)c1oc(-c2cc(Cl)cc(Cl)c2Cl)nc1C#N ; C#CCNc1oc(-c2cc(Cl)cc(Cl)c2Cl)nc1C#N ; CNc1sc(-c2cc(Cl)cc(Cl)c2Cl)nc1C#N]

or a pharmaceutically acceptable salt thereof.

In certain embodiments, lipoxygenase inhibitors include compounds of Formula (II), such as described in U.S. Provisional Patent Application No. 63/231,061 filed on Aug. 9, 2021, the disclosure of which is herein incorporated by reference.

In some embodiments, the lipoxygenase inhibitor in compositions of interest is a compound of formula of Formula (III):

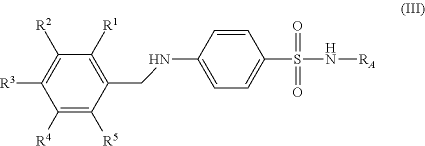

where R¹, R², R³, R⁴ and R⁵ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
R_{A} is cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl,
or a salt, solvate or hydrate thereof.

In some embodiments, R¹ is hydroxy. In some embodiments, R² is alkoxy. In some instances, R² is a C1-C12 alkoxy. In some instances, R² is selected from methoxy, ethoxy, propoxy, butoxy, isobutoxy and tert-butoxy. In certain instances, R² is methoxy. In some instances, R³ is hydrogen. In some instances, R⁴ is hydrogen. In some instances, R⁵ is hydrogen. In some instances, each of R³, R⁴ and R⁵ are hydrogen.

In some embodiments, R_{A} is selected from the group consisting of 2-benzothiazole, 2-benzoxazole, 2-benzimidazole, 2-thiophene, 4-methyl-2-benzothiazole, 4-methyl-2-thiazole, 5-methyl-2-thiazole, 5-phenyl-2-thiazole, 4,5-methyl-2-thiazole, 5-methyl-3-isoxazole, 3-methoxy-phenyl, 3-quinolone, 8-isoquinolone, phenyl, 1-naphthalene, 2-naphthalene, 1,4-bi-phenyl, 1.3-bi-phenyl, 3-piperazine-phenyl, 4-piperazine-phenyl, 4-piperidine-phenyl, 4-piperazine-3-pyridine, 6-methyl-3-pyridine, 2-pyridine, 3-pyridine, 2-pyrimidine, 3-tert-butyl-phenyl, 6-methoxy-2-benzothiazole, 4-phenyl-2-thiazole, 3-morpholine-phenyl, 4N-boc-piperidine-3-phenyl, 3-piperidine-phenyl, 3-isopropyl-phenyl and 6-F-2-benzothiazole.

In some embodiments, the lipoxygenase inhibitor in compositions of interest is a compound of formula of Formula (IIIA):

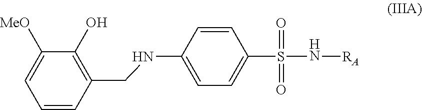

where R_{A} is selected from the group consisting of 2-benzothiazole, 2-benzoxazole, 2-benzimidazole, 2-thiophene, 4-methyl-2-benzothiazole, 4-methyl-2-thiazole, 5-methyl-2-thiazole, 5-phenyl-2-thiazole, 4,5-methyl-2-thiazole, 5-methyl-3-isoxazole, 3-methoxy-phenyl, 3-quinolone, 8-isoquinolone, phenyl, 1-naphthalene, 2-naphthalene, 1,4-bi-phenyl, 1.3-bi-phenyl, 3-piperazine-phenyl, 4-piperazine-phenyl, 4-piperidine-phenyl, 4-piperazine-3-pyridine, 6-methyl-3-pyridine, 2-pyridine, 3-pyridine, 2-pyrimidine, 3-tert-butyl-phenyl, 6-methoxy-2-benzothiazole, 4-phenyl-2-thiazole, 3-morpholine-phenyl, 4N-boc-piperidine-3-phenyl, 3-piperidine-phenyl, 3-isopropyl-phenyl and 6-F-2-benzothiazole.

In certain embodiments, the lipoxygenase inhibitor in compositions of interest is a compound of formula of Formula (IIIB):

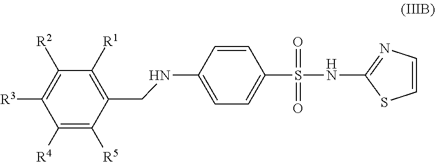

where R¹, R², R³, R⁴ and R⁵ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl.

In some embodiments, R¹, R², R³, R⁴ and R⁵ are each hydrogen. In some embodiments, R¹ is hydroxy. In some embodiments, R² is alkoxy. In some embodiments, R³ is alkoxy. In some instances, R² is a C1-C12 alkoxy. In some instances, R² is selected from methoxy, ethoxy, propoxy, butoxy, isobutoxy and tert-butoxy. In certain instances, R² is methoxy. In some instances, R³ is hydrogen. In some instances, R³ is a C₁-C₁₂ alkoxy. In some instances, R³ is selected from methoxy, ethoxy, propoxy, butoxy, isobutoxy and tert-butoxy. In certain instances, R³ is methoxy. In some instances, R⁴ is hydrogen. In some instances, R⁴ is a C₁-C₁₂ alkoxy. In some instances, R⁴ is selected from methoxy, ethoxy, propoxy, butoxy, isobutoxy and tert-butoxy. In certain instances, R⁴ is methoxy. In some instances, R⁵ is hydrogen. In some instances, each of R³, R⁴ and R⁵ are hydrogen. In some embodiments, R¹, R², R³, R⁴ and R⁵ are each independently selected from a halogen such as Cl, F or Br, methyl, amino, nitro, allyl and methoxy.

In certain embodiments, the lipoxygenase inhibitor in compositions of interest is a compound of formula of Formula (IIIC):

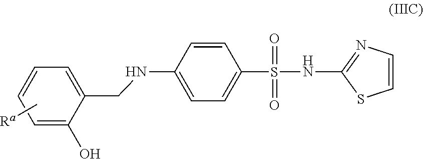

where R^{a} is selected from 2-amino-Ph; 3-OH-Ph; 2-amino-3-methoxy-Ph; 3-OH-4-methoxy-Ph; 7-indole; 2,3-Cl-Ph; 3-Cl; 3-F; 3-Br; 4-Br; 3-methyl; 3-amino; 3-nitro; 3-allyl; 4-Cl; 4-methoxy; 5-Cl; 5-methoxy; 5-nitro; 5-F; 5-nitro and 6-methoxy.

In certain embodiments, the lipoxygenase inhibitor in compositions of interest is a compound of formula of Formula (IIID):

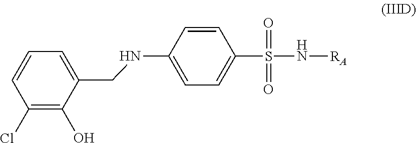

where R_{A} is selected from the group consisting of phenyl, 1-naphthalene, 2-benzothiazole, 2-naphthalene, 4-biphenyl, 8-isoquinoline, 3-quinoline and 4-piperidine-phenyl.

In certain embodiments, the lipoxygenase inhibitor in compositions of interest is a compound of formula of Formula (IIIE):

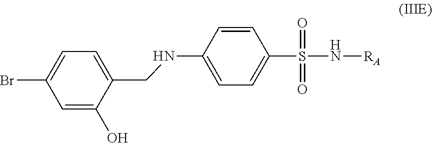

where R_{A} is selected from the group consisting of phenyl, 1-naphthalene, 2-benzothiazole, 3-quinoline, 2-naphthalene, 4-biphenyl and 8-quinoline.

In certain embodiments, the lipoxygenase inhibitor in compositions of interest is a compound of formula of Formula (IIIF):

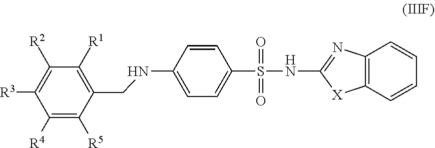

where R¹, R², R³, R⁴ and R⁵ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
X is S or O
or a salt, solvate or hydrate thereof.

In some embodiments, R¹, R², R³, R⁴ and R⁵ are each hydrogen. In some embodiments, R¹ is hydroxy. In some embodiments, R² is alkoxy. In some embodiments, R³ is alkoxy. In some instances, R² is a C1-C12 alkoxy. In some instances, R² is selected from methoxy, ethoxy, propoxy, butoxy, isobutoxy and tert-butoxy. In certain instances, R² is methoxy. In some instances, R³ is hydrogen. In some instances, R³ is a C1-C12 alkoxy. In some instances, R³ is selected from methoxy, ethoxy, propoxy, butoxy, isobutoxy and tert-butoxy. In certain instances, R³ is methoxy. In some instances, R⁴ is hydrogen. In some instances, R⁴ is a C1-C12 alkoxy. In some instances, R⁴ is selected from methoxy, ethoxy, propoxy, butoxy, isobutoxy and tert-butoxy. In certain instances, R⁴ is methoxy. In some instances, R⁵ is hydrogen. In some instances, each of R³, R⁴ and R⁵ are hydrogen. In some embodiments, R¹, R², R³, R⁴ and R⁵ are each independently selected from a halogen such as Cl, F or Br, methyl, amino, nitro, allyl and methoxy.

In some embodiments, X is oxygen. In other embodiments, X is S.

In certain embodiments, the lipoxygenase inhibitor in compositions of interest is a compound of formula of Formula (IIIG):

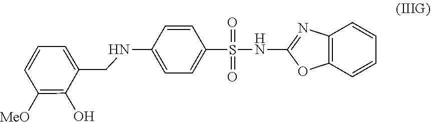 [SMILES: COc1cccc(CNc2ccc(S(=O)(=O)Nc3nc4ccccc4o3)cc2)c1O]

In certain embodiments, the lipoxygenase inhibitor in compositions of interest is a compound of formula of Formula (IIIH):

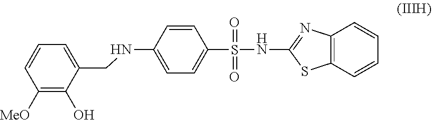 [SMILES: COc1cccc(CNc2ccc(S(=O)(=O)Nc3nc4ccccc4s3)cc2)c1O]

In some embodiments, the lipoxygenase inhibitor in compositions of interest is a compound of formula of Formula (IV):

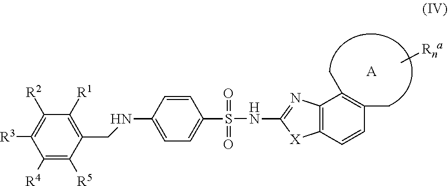

where R¹, R², R³, R⁴ and R⁵ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
X is S or O;
the A ring is absent or when the A ring is present, the A ring is a substituted or unsubstituted 5 to 12 membered ring;
n is an integer from 0 to 12; and
each R^{a} is independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl,
or a salt, solvate or hydrate thereof.

In some embodiments, R¹ is hydroxy. In some embodiments, R² is alkoxy. In some instances, R² is a C1-C12 alkoxy. In some instances, R² is selected from methoxy, ethoxy, propoxy, butoxy, isobutoxy and tert-butoxy. In certain instances, R² is methoxy. In some instances, R³ is hydrogen. In some instances, R⁴ is hydrogen. In some instances, R⁵ is hydrogen.

In some instances, each of R³, R⁴ and R⁵ are hydrogen.

In some embodiments, X is oxygen. In other embodiments, X is S.

In some embodiments, A is a six-membered ring. In some instances, A is a six-membered heterocyclic ring. In some instances, A is a six-membered aryl ring. In some instances, A is a six-membered heteroaryl ring. In some embodiments, A is a six-membered ring and the compound is of formula (IA):

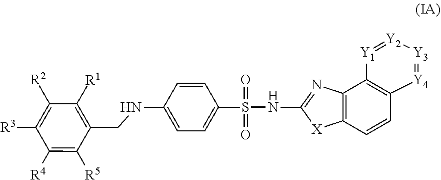

where Y₁, Y₂, Y₃ and Y₄ are each independently C or N. In some embodiments, Y₁, Y₂, Y₃ and Y₄ are each C. In some embodiments, Y₃ is N and Y₁, Y₂, and Y₄ are each C. In some embodiments, Y₁ and Y₄ are N; and Y₂ and Y₃ are C. In some embodiments, A is unsubstituted.

In some embodiments, A is monosubstituted. In other embodiments, A is di-substituted. In other embodiments, A is tri-substituted. In some instances, n is 1 or 2 and each R^{a} is independently selected from:

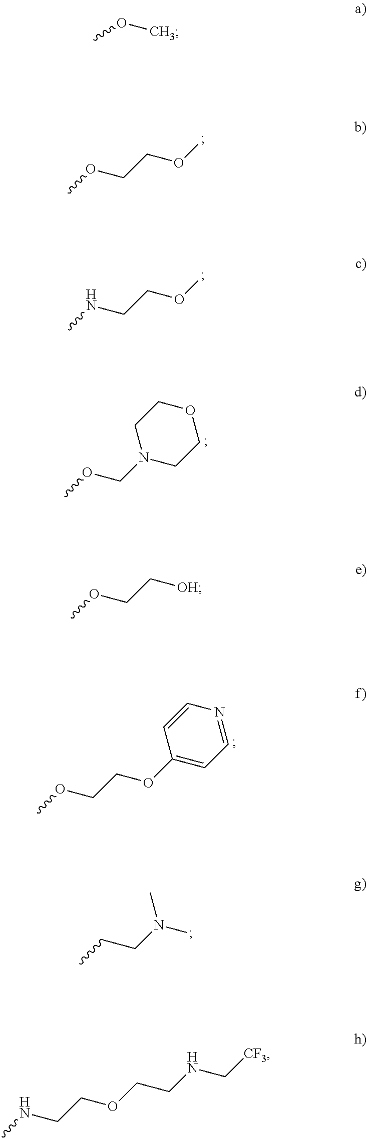

where [symbol] represents the A-R^{a} bond.

In some embodiments, A is a five-membered ring. In some instances, A is a five-membered heterocyclic ring. In some instances, A is a five-membered heteroaryl ring. In some embodiments, A is a five-membered ring and the compound is of formula (IB):

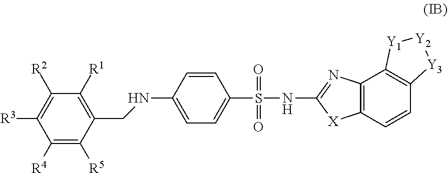

where Y₁, Y₂, Y₃ and Y₄ are each independently C, N or O.

In some embodiments, Y₁, Y₂ and Y₃ are each C. In some embodiments, Y₂ is C and Y₁, and Y₃ are each O. In some embodiments, Y₁ is N; and Y₂ and Y₃ are C. In some embodiments, Y₃ is N; and Y₁ and Y₂ are C. In some embodiments, Y₁ is O; Y₂ is C and Y₃ is N. In some embodiments, Y₁ is N; Y₂ is C and Y₃ is O. In some embodiments, A is unsubstituted. In some embodiments, A is monosubstituted. In other embodiments, A is di-substituted. In other embodiments, A is tri-substituted. In some instances, n is 1 or 2 and each R^{a} is independently selected from:

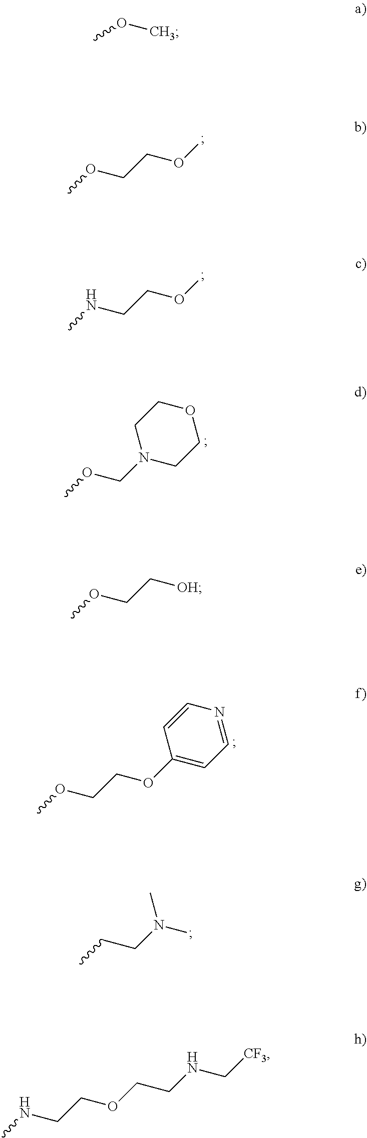

where [symbol] represents the A-R^{a} bond.

In certain embodiments, the compound is 4-((2-hydroxy-3-methoxybenzyl)amino)-N-(naphtho[1,2-d]thiazol-2-yl)benzenesulfonamide:

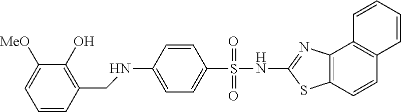 [SMILES: COc1cccc(CNc2ccc(S(=O)(=O)Nc3nc4c(ccc5ccccc54)s3)cc2)c1O]

or a pharmaceutically acceptable salt, solvate or hydrate thereof.

In certain embodiments, lipoxygenase inhibitors include compounds such as described in U.S. Provisional Patent Application No. 63/227,061 filed on Jul. 30, 2021, and International Patent Application No. PCT/US2022/28119, filed on May 6, 2022, the disclosures of which are herein incorporated by reference.

Aspects of the present disclosure also include compositions having a pharmaceutically acceptable carrier and one or more of the compounds described above. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7th ed., Lippincott, Williams, & Wilkins; and Handbook of Pharmaceutical Excipients (2000) A. H. Kibbe et al., eds., 3rd ed. Amer. Pharmaceutical Assoc. For example, the one or more excipients may include sucrose, starch, mannitol, sorbitol, lactose, glucose, cellulose, talc, calcium phosphate or calcium carbonate, a binder (e.g., cellulose, methylcellulose, hydroxymethylcellulose, polypropylpyrrolidone, polyvinylpyrrolidone, gelatin, gum arabic, poly(ethylene glycol), sucrose or starch), a disintegrator (e.g., starch, carboxymethylcellulose, hydroxypropyl starch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinylpyrrolidone or aluminum stearate), a dispersing agent (e.g., hydroxypropylmethylcellulose), a diluent (e.g., water), and base wax (e.g., cocoa butter, white petrolatum or polyethylene glycol).

The compounds may be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. In certain embodiments, the conjugate compounds are formulated for injection. For example, compositions of interest may be formulated for intravenous or intraperitoneal administration.

In pharmaceutical dosage forms, the compounds may be administered in the form of its pharmaceutically acceptable salts, or it may also be used alone or in appropriate association, as well as in combination, with other pharmaceutically active compounds. The following methods and excipients are merely exemplary and are in no way limiting.

In some embodiments, compositions of interest include an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. In some instances, compositions of interest further include a preservative. Suitable preservatives include, but are not limited to, a benzyl alcohol, phenol, chlorobutanol, benzalkonium chloride, and the like. In many cases, the composition is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

In some embodiments, compositions include other additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Where the composition is formulated for injection, the compounds may be formulated by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Although the dosage used in treating a subject will vary depending on the clinical goals to be achieved, a suitable dosage range of the compound is one which provides up to about 0.0001 mg to about 5000 mg, e.g., from about 1 mg to about 25 mg, from about 25 mg to about 50 mg, from about 50 mg to about 100 mg, from about 100 mg to about 200 mg, from about 200 mg to about 250 mg, from about 250 mg to about 500 mg, from about 500 mg to about 1000 mg, or from about 1000 mg to about 5000 mg of an active agent, which can be administered in a single dose. Those of skill will readily appreciate that dose levels can vary as a function of the specific compound, the severity of the symptoms and the susceptibility of the subject to side effects.

In some embodiments, a single dose of the compound is administered. In other embodiments, multiple doses of the compound are administered. Where multiple doses are administered over a period of time, the compound may be administered, e.g., twice daily (qid), daily (qd), every other day (qod), every third day, three times per week (tiw), or twice per week (biw) over a period of time. For example, the compound may be administered qid, qd, qod, tiw, or biw over a period of from one day to about 2 years or more. For example, the compound may be administered at any of the aforementioned frequencies for one week, two weeks, one month, two months, six months, one year, or two years, or more, depending on various factors.

Dose units of the present disclosure can be made using manufacturing methods available in the art and can be of a variety of forms suitable for injection (including topical, intracisternal, intrathecal, intravenous, intramuscular, subcutaneous and dermal) administration, for example as a solution, suspension, solution, lyophilate or emulsion. The dose unit can contain components conventional in pharmaceutical preparations, e.g. one or more carriers, binders, lubricants, excipients (e.g., to impart controlled release characteristics), pH modifiers, coloring agents or further active agents.

Dose units can comprise components in any relative amounts. For example, dose units can be from about 0.1% to 99% by weight of active ingredients (i.e., compounds described herein) per total weight of dose unit. In some embodiments, dose units can be from 10% to 50%, from 20% to 40%, or about 30% by weight of active ingredients per total weight dose unit.

### Methods for Modulating a Lipoxygenase Enzyme

As summarized above, aspects of the present disclosure also modulating or inhibiting a lipoxygenase enzyme, such as human reticulocyte 15-lipoxygenase-1. In some embodiments, methods include contacting a cell having a lipoxygenase enzyme (e.g., 12/15-lipoxygenase) with one or more of the compositions described herein in vitro. In other embodiments, methods include contacting a cell with one or more of the compositions described herein in vivo (e.g., by administering to a subject as described in greater detail below). In still other embodiments a cell is contacted ex vivo. In some embodiments, methods include decreasing or reducing lipoxygenase activity, such as reducing lipoxygenase activity by 1% or more, such as by 5% or more, such as by 10% or more, such as by 15% or more, such as by 20% or more, such as by 25% or more, such as by 30% or more, such as by 35% or more, such as by 40% or more, such as by 45% or more, such as by 50% or more, such as by 60% or more, such as by 70% or more, such as by 80% or more, such as by 90% or more, such as by 95% or more, such as by 97% or more, such as by 99% or more and including by 99.9% or more.

In some embodiments, the subject methods include modulating the generation of hydroperoxy eicosatetraeneoic acids (HpETEs) in cells, such as where generation of hydroperoxy eicosatetraeneoic acids (e.g., HpETE-PE) is reduced by 1% or more, such as by 5% or more, such as by 10% or more, such as by 15% or more, such as by 20% or more, such as by 25% or more, such as by 30% or more, such as by 35% or more, such as by 40% or more, such as by 45% or more, such as by 50% or more, such as by 60% or more, such as by 70% or more, such as by 80% or more, such as by 90% or more, such as by 95% or more, such as by 97% or more, such as by 99% or more and including by 99.9% or more. In certain embodiments, methods include modulating ferroptosis. In certain instances, methods include contacting one or more of the compositions described herein with cells having lipoxygenase in a manner sufficient to reduce the accumulation of hydroperoxy membrane phospholipids in the contacted cells by 1% or more, such as by 5% or more, such as by 10% or more, such as by 15% or more, such as by 20% or more, such as by 25% or more, such as by 30% or more, scuh as by 35% or more, such as by 40% or more, such as by 45% or more, such as by 50% or more, such as by 60% or more, such as by 70% or more, such as by 80% or more, such as by 90% or more, such as by 95% or more, such as by 97% or more, such as by 99% or more and including by 99.9% or more.

In other embodiments, methods include modulating or reducing modulating eicosanoid mediator biosynthesis from leukotrienes (LTs) to pro-resolving mediator class of lipoxins (LXs). In some embodiments, methods include modulating h15-LOX-2 in a manner sufficient to reduce foam cell formation and atherosclerotic plaque accumulation, such as by 1% or more, such as by 5% or more, such as by 10% or more, such as by 15% or more, such as by 20% or more, such as by 25% or more, such as by 30% or more, such as by 35% or more, such as by 40% or more, such as by 45% or more, such as by 50% or more, such as by 60% or more, such as by 70% or more, such as by 80% or more, such as by 90% or more, such as by 95% or more, such as by 97% or more, such as by 99% or more and including by 99.9% or more.

In some instances, methods include treating or preventing a lipoxygenase mediated disease. The term “treat” or “treatment” of any condition, refers, in certain embodiments, to ameliorating the condition (i.e., arresting or reducing the development of the condition). In certain embodiments “treating” or “treatment” refers to ameliorating at least one physical parameter, which may not be discernible by the patient. In certain embodiments, “treating” or “treatment” refers to inhibiting the condition, either physically, (e.g., stabilization of a discernible symptom), physiologically, (e.g., stabilization of a physical parameter), or both. In certain embodiments, “treating” or “treatment” refers to delaying the onset of the condition. The term “therapeutically effective amount” is used herein to refer to the amount of a compound that, when administered to a patient for preventing or treating a condition is sufficient to effect such treatment. The “therapeutically effective amount” will vary depending on the compound, the condition and its severity and the age, weight, etc., of the patient.

In practicing the subject methods, a therapeutically effective amount of one or more of the compositions disclosed herein is administered to a subject sufficient to treat or prevent the lipoxygenase-mediated diseases. In embodiments, the term “subject” is meant the person or organism to which the compound is administered. As such, subjects of the present disclosure may include but are not limited to mammals, e.g., humans and other primates, such as chimpanzees and other apes and monkey species, dogs, rabbits, cats and other domesticated pets; and the like, where in certain embodiments the subject are humans. The term “subject” is also meant to include a person or organism of any age, weight or other physical characteristic, where the subjects may be an adult, a child, an infant or a newborn.

In some instances, the lipoxygenase-mediated disease is a cardiovascular disease or condition such as atherosclerotic plaque formation or accumulation. In some embodiments, methods include treating a subject for cystic fibrosis lung disease. In some embodiments, methods include treating a subject for neurodegenerative disease. In certain instances, methods include treating a subject for Alzheimer's disease. In certain instances, methods include treating a subject for Parkinson's disease. In certain instances, methods include treating a subject for Huntington's disease. In certain embodiments, methods further include diagnosing the subject as having one or more a cardiovascular disease, cystic fibrosis lung disease or a neurodegenerative disease such as Alzheimer's disease, Parkinson's disease, and Huntington's disease. In some embodiments, methods include administering one or more of the compounds described herein to a subject diagnosed with one or more a cardiovascular disease, cystic fibrosis lung disease and a neurodegenerative disease such as Alzheimer's disease, Parkinson's disease, and Huntington's disease. In certain embodiments, methods include treating a subject that has or is diagnosed as having a disease or disorder selected from: stroke, diabetes, obesity, asthma, glomerulonephritis, osteoporosis, periventricular leukomalacia, cardiac arrest with resuscitation, atherosclerosis, neurodegenerative or neuroinflammatory disorders selected from Parkinson's disease, Alzheimer's disease, Huntington's disease and dementia, cancer, brain injury, a disease involving hypoxia or anoxia, myocardial infarction, cardiovascular disease, chronic heart failure, congestive heart, ischemia selected from cerebral ischemia, retinal ischemia, myocardial ischemia, or post-surgical cognitive dysfunction, inflammatory disease, arterial inflammation, inflammatory bowel disease, Crohn's disease, renal disease, asthma, allergic rhinitis, gout, cardiopulmonary inflammation, rheumatoid arthritis, osteoarthritis, muscle fatigue, acne, dermatitis, or psoriasis), chronic bronchitis, mucus hypersecretion, chronic obstructive pulmonary disease (COPD), pulmonary fibrosis, fibrosis caused by chemotherapy, idiopathic pulmonary fibrosis, cystic fibrosis, adult respiratory distress syndrome, CNS disorders, psychiatric disorders, anxiety, depression, peripheral neuropathy, spinal cord injury, head injury, surgical trauma, allograft tissue or organ transplant rejection, autoimmune disorder, eczema and disorders involving bone loss or bone formation. In certain embodiments, methods include treating a subject for stroke.

Compositions as described herein may be administered to a subject by any convenient protocol, including, but not limited, to intraperitoneally, topically, orally, sublingually, parenterally, intravenously, vaginally, rectally as well as by transdermal protocols. In certain embodiments, the subject compounds are administered by intravenous injection. In certain embodiments, the subject compounds are administered by intraperitoneal injection.

Depending on the condition being treated, the amount of lipoxygenase inhibitor in the composition administered to the subject may vary, such as ranging from about 0.0001 mg/day to about 10,000 mg/day, such as from about 0.001 mg/day to about 9000 mg/day, such as from 0.01 mg/day to about 8000 mg/day, such as from about 0.1 mg/day to about 7000 mg/day, such as from about 1 mg/day to about 6000 mg/day, including from about 5 mg/day to about 5000 mg/day. Each dosage of the compound or pharmaceutically acceptable salt administered to the subject may vary ranging from about 1 mg/kg to about 1000 mg/kg, such as from about 2 mg/kg to about 900 mg/kg, such as from about 3 mg/kg to about 800 mg/kg, such as from about 4 mg/kg to about 700 mg/kg, such as from 5 mg/kg to about 600 mg/kg, such as from 6 mg/kg to about 500 mg/kg, such as from 7 mg/kg to about 400 mg/kg, such as from about 8 mg/kg to about 300 mg/kg, such as from about 9 mg/kg to about 200 mg/kg and including from about 10 mg/kg to about 100 mg/kg.

In certain embodiments, protocols may include multiple dosage intervals. By “multiple dosage intervals” is meant that two or more dosages of the compound is administered to the subject in a sequential manner. In practicing methods of the present disclosure, treatment regimens may include two or more dosage intervals, such as three or more dosage intervals, such as four or more dosage intervals, such as five or more dosage intervals, including ten or more dosage intervals. The duration between dosage intervals in a multiple dosage interval treatment protocol may vary, depending on the physiology of the subject or by the treatment protocol as determined by a health care professional. For example, the duration between dosage intervals in a multiple dosage treatment protocol may be predetermined and follow at regular intervals. As such, the time between dosage intervals may vary and may be 1 day or longer, such as 2 days or longer, such as 4 days or longer, such as 6 days or longer, such as 8 days or longer, such as 12 days or longer, such as 16 days or longer and including 24 days or longer. In certain embodiments, multiple dosage interval protocols provide for a time between dosage intervals of 1 week or longer, such as 2 weeks or longer, such as 3 weeks or longer, such as 4 weeks or longer, such as 5 weeks or longer, including 6 weeks or longer.

The cycles of drug administration may be repeated for 1, 2, 3, 4, 5, 6, 7, 8 or more than 8 dosage cycles, for a total period of 6 months or 1 year or 2 years or 3 years or 4 years or more. In certain embodiments, one or more of the subject compounds are administered for the rest of the subject's lifetime.

In certain embodiments, compounds of the present disclosure can be administered prior to, concurrent with, or subsequent to other therapeutic agents for treating the same or an unrelated condition. If provided at the same time as another therapeutic agent, the present compounds may be administered in the same or in a different composition. Thus, the compounds of interest and other therapeutic agents can be administered to the subject by way of concurrent therapy. By “concurrent therapy” is intended administration to a subject such that the therapeutic effect of the combination of the substances is caused in the subject undergoing therapy. For example, concurrent therapy may be achieved by administering the compounds of the present disclosure with a pharmaceutical composition having at least one other agent, such as an anti-inflammatory agent, immunosuppressant, steroid, analgesic, anesthetic, antihypertensive, chemotherapeutic, among other types of therapeutics, which in combination make up a therapeutically effective dose, according to a particular dosing regimen. Administration of the separate pharmaceutical compositions can be performed simultaneously or at different times (i.e., sequentially, in either order, on the same day, or on different days), so long as the therapeutic effect of the combination of these substances is caused in the subject undergoing therapy.

Where the compositions of the present disclosure is administered concurrently with a second therapeutic agent to treat the same condition (e.g., an anti-atherosclerotic drug, etc.) the weight ratio of the lipoxygenase inhibitor in the composition to second therapeutic agent may range from 1:2 and 1:2.5; 1:2.5 and 1:3; 1:3 and 1:3.5 1:3.5 and 1:4; 1:4 and 1:4.5; 1:4.5 and 1:5; 1:5 and 1:10; and 1:10 and 1:25 or a range thereof. For example, the weight ratio of the subject compound to second therapeutic agent may range between 1:1 and 1:5; 1:5 and 1:10; 1:10 and 1:15; or 1:15 and 1:25. Alternatively, the weight ratio of the second therapeutic agent to the subject compound ranges between 2:1 and 2.5:1; 2.5:1 and 3:1; 3:1 and 3.5:1; 3.5:1 and 4:1; 4:1 and 4.5:1; 4.5:1 and 5:1; 5:1 and 10:1; and 10:1 and 25:1 or a range thereof. For example, the ratio of the second therapeutic agent the subject compound may range between 1:1 and 5:1; 5:1 and 10:1; 10:1 and 15:1; or 15:1 and 25:1.

In certain embodiments, methods include formulating and administering a composition as is described in U.S. Pat. No. 10,287,279 and U.S. Provisional Patent Application No. 63/231,061 filed on Aug. 9, 2021, the disclosures of which are herein incorporated by reference.

Aspects, including embodiments, of the subject matter described herein may be beneficial alone or in combination, with one or more other aspects or embodiments. Without limiting the description, certain non-limiting aspects of the disclosure numbered 1-72 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A composition comprising:
a solvent component comprising:
   a first solvent selected from the group consisting of dimethylacetamide (DMA), diethylacetamide (DEA), dimethylsulfoxide (DMSO), a polyoxyethylene ester of 12-hydroxysteraric acid, a polyethoxylated castor oil, a polyvinyl caprolactam-polyvinyl acetate-polyethylene glycol, a propylene glycol caprylate, a diethylene glycol monomethyl ether, a caprylocaproyl macrogolglyceride, a polyoxyl-6 glyceride and a propylene glycol monolaurate; and
   a second solvent comprising a polyethylene glycol; and
a lipoxygenase inhibitor,
wherein the lipoxygenase inhibitor is present in the composition at a concentration of 30 mg/mL or greater.
   2. The composition according to 1, wherein the composition is formulated for intravenous administration to a subject.
   3. The composition according to 2, wherein the composition is formulated for bolus injection or sustained infusion.
   4. The composition according to any one of 1-3, wherein the polyoxyethylene ester of 12-hydroxysteraric acid comprises a solvent that is or is substantially the same as Solutol.
   5. The composition according to any one of 1-3, wherein the polyethoxylated castor oil comprises a solvent that is or is substantially the same as Kolliphor.
   6. The composition according to any one of 1-3, wherein the solvent comprises a solvent that is or is substantially the same as Kolliphor EL.
   7. The composition according to any one of 1-3, wherein the solvent comprises a solvent that is or is substantially the same as Kolliphor RH40.
   8. The composition according to any one of 1-3, wherein the solvent comprises a solvent that is or is substantially the same as Kolliphor P188 Geismar.
   9. The composition according to any one of 1-3, wherein the polyvinyl caprolactam-polyvinyl acetate-polyethylene glycol comprises a solvent that is or is substantially the same as Soluplus.
   10. The composition according to any one of 1-3, wherein the propylene glycol caprylate comprises a solvent that is or is substantially the same as Capryol.
   11. The composition according to any one of 1-3, wherein the diethylene glycol monomethyl ether comprises a solvent that is or is substantially the same as Transcutol.
   12. The composition according to any one of 1-3, wherein the caprylocaproyl macrogolglyceride comprises a solvent that is or is substantially the same as Labrasol.
   13. The composition according to any one of 1-3, wherein the polyoxyl-6 glyceride comprises a solvent that is or is substantially the same as Labrafil.
   14. The composition according to any one of 1-3, wherein the propylene glycol monolaurate comprises a solvent that is or is substantially the same as Lauroglycol.
   15. The composition according to any one of 1-14, wherein the second solvent comprises polyethylene glycol 400.
   16. The composition according to any one of 1-5, wherein the first solvent and second solvent are present in the composition at a ratio of from 5:95 to 50:50.
   17. The composition according to 16, wherein the first solvent and second solvent are present in the composition at a ratio of about 5:95.
   18. The composition according to 16, wherein the first solvent and second solvent are present in the composition at a ratio of about 10:90.
   19. The composition according to 16, wherein the first solvent and second solvent are present in the composition at a ratio of about 15:85.
   20. The composition according to 16, wherein the first solvent and second solvent are present in the composition at a ratio of about 20:80.
   21. The composition according to any one of 1-20, wherein the lipoxygenase inhibitor is present in the composition at a concentration of 50 mg/mL or more.
   22. The composition according to any one of 1-20, wherein the lipoxygenase inhibitor is present in the composition at a concentration of 75 mg/mL or more.
   23. The composition according to any one of 1-20, wherein the lipoxygenase inhibitor is present in the composition at a concentration of 100 mg/mL or more.
   24. The composition according to any one of 1-20, wherein the lipoxygenase inhibitor is present in the composition at a concentration of 125 mg/mL or more.
   25. The composition according to any one of 1-20, wherein the lipoxygenase inhibitor is present in the composition at a concentration of 150 mg/mL or more.
   26. The composition according to any one of 1-25, wherein the lipoxygenase inhibitor is a compound of Formula (I):

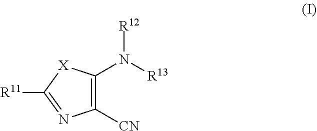

or a pharmaceutically acceptable salt thereof, in which:
X is O or S;
R¹¹ is an aryl, heteroaryl, cyclyl, or heterocyclyl, each of which can be optionally substituted; and
R¹² and R¹³ are independently hydrogen, halogen, alkyl, alkenyl, alkynyl, aralkyl, acyl, aryl, heteroaryl, cyclyl or heterocyclyl, each of which can be optionally substituted or a pharmaceutically acceptable salt thereof.
   27. The composition according to 26, wherein the lipoxygenase inhibitor is a compound of Formula (Ia):

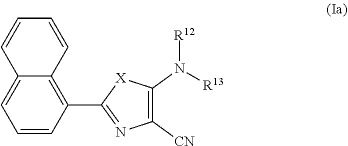

or a pharmaceutically acceptable salt thereof, in which:
X is O or S;
R¹² and R¹³ are independently hydrogen, halogen, alkyl, alkenyl, alkynyl, aralkyl, acyl, aryl, heteroaryl, cyclyl or heterocyclyl, each of which can be optionally substituted
or a pharmaceutically acceptable salt thereof.
   28. The composition according to 27, wherein X is O.
   29. The composition according to 27, wherein the compound is selected from the group consisting of: 5-(methylamino)-2-naphthalen-1-yl-1,3-oxazole-4-carbonitrile (ML351), 2-(2,3-dichlorophenyl)-5-(methylamino)-1,3-oxazole-4-carbonitrile, 2-(3,4-dichlorophenyl)-5-(methylamino)-1,3-oxazole-4-carbonitrile, 5-(methylamino)-2-naphthalen-1-yl-1,3-thiazole-4-carbonitrile or a pharmaceutically acceptable salt thereof.
   30. The composition according to any one of 1-25, wherein the lipoxygenase inhibitor is a compound of Formula (II):

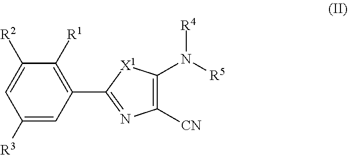

or a pharmaceutically acceptable salt thereof, wherein:
X¹ is selected from O and S;
R¹, R², and R³ are each independently selected from halo, CN, C₁₋₃ alkyl, C₁₋₃ haloalkyl, C₁₋₃ alkoxy, and C₁₋₃ haloalkoxy;
R⁴ is selected from H, C₁₋₃ alkyl, and HO-CL-3 alkylene;
R⁵ is selected from C₁₋₆ alkyl, C₂₋₆ alkenyl, C₂₋₆ alkynyl, C(O)OR^{a1}, C(O)N(R^{a1})₂, P(═O)(OR^{a1})₂, and C(O)R^{b1}; wherein said C₁₋₆ alkyl, C₂₋₆ alkenyl, and C₂₋₆ alkynyl are each optionally substituted with a substituent selected from OR^{a1} and OP(═O)(OR^{a1})₂;
each R^{a1} is independently selected from H, C₁₋₆ alkyl, C₆₋₁₀ aryl, C₁₋₆ alkyl-C₆₋₁₀ aryl, and C₁₋₆ alkyl-C₆₋₁₀ aryl-C₁₋₆ alkyl, wherein said C₁₋₆ alkyl, C₆₋₁₀ aryl, C₁₋₆ alkyl-C₆₋₁₀ aryl, and C₁₋₆ alkyl-C₆₋₁₀ aryl-C₁₋₆ alkyl are each optionally substituted with a substituent selected from amino, C₁₋₆ alkylamino, (C₁₋₆ haloalkyl)amino, di(C₁₋₆ alkyl)amino, (C₁₋₆ alkyl)(C₁₋₆ haloalkyl)amino, (C₆₋₁₀ aryl)amino, (C₆₋₁₀ aryl)(C₁₋₆ alkyl)amino, (5-6-membered heteroaryl)amino, (5-6-membered heteroaryl)(C₁₋₆ alkyl)amino, C₆₋₁₀ aryl, 4-6 membered heterocycloalkyl, 5-6-membered heteroaryl, and OR², wherein said C₆₋₁₀ aryl, 4-6 membered heterocycloalkyl, and 5-6-membered heteroaryl are each optionally substituted with 1, 2, or 3 substituents independently selected from amino, C₁₋₆ alkylamino, di(C₁₋₆ alkyl)amino, carboxy, and halo;
each R^{a2} is independently selected from H, C₁₋₃ alkyl, C₁₋₃ haloalkyl, C₁₋₃ alkoxy-C₁₋₃ alkyl, 4-7 membered heterocycloalkyl-C₁₋₃ alkyl, 5-6-membered heteroaryloxy-C₁₋₃ alkyl, C₆₋₁₀ aryl, and 5-6-membered heteroaryl, wherein said C₆₋₁₀ aryl and 5-6-membered heteroaryl are each optionally substituted with 1, 2, or 3 substituents independently selected from halo, C₁₋₃ alkoxy, C₁₋₃ haloalkoxy, C₁₋₃ alkyl, and C₁₋₃ haloalkyl; and
R^{b1} is C₁₋₆ alkyl, optionally substituted with a substituent selected from amino, C₁₋₆ alkylamino, di(C₁₋₆ alkyl)amino, and 4-7 membered heterocycloalkyl ring comprising at least one N atom,
or a pharmaceutically acceptable salt thereof.
   31. The composition according to 30, wherein the lipoxygenase inhibitor is selected from the group consisting of:

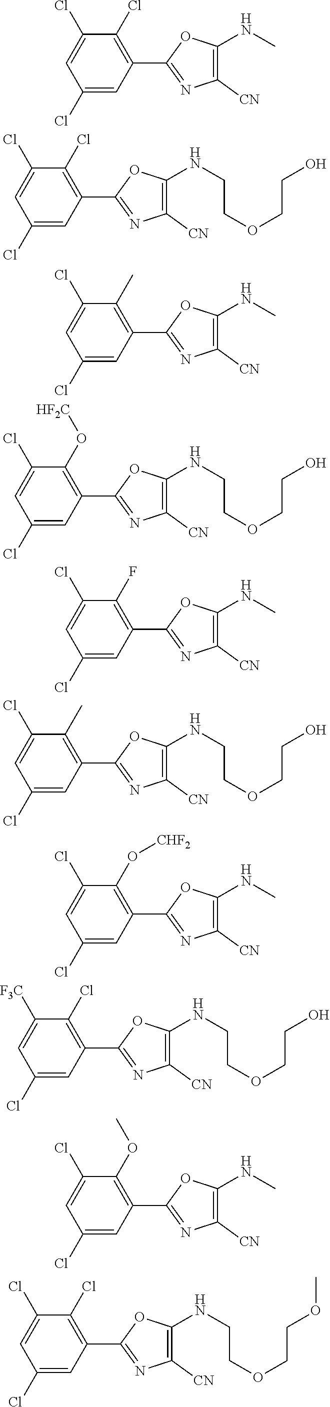

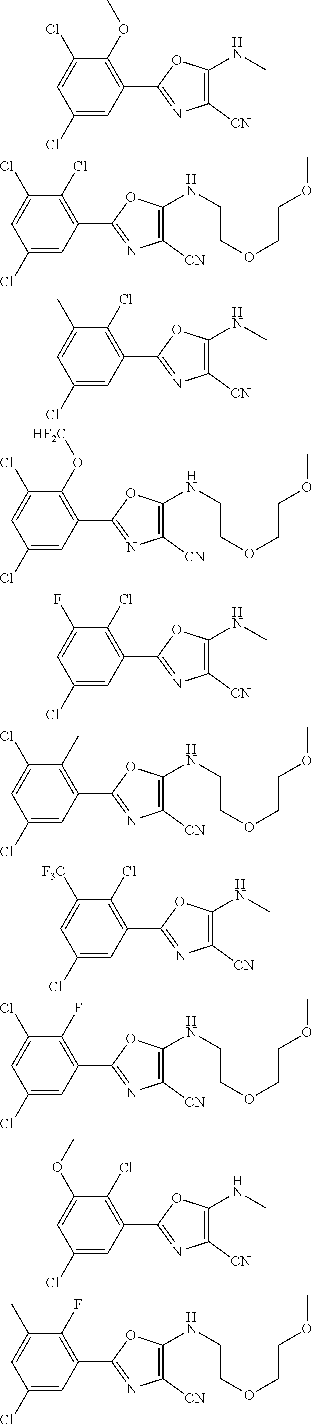

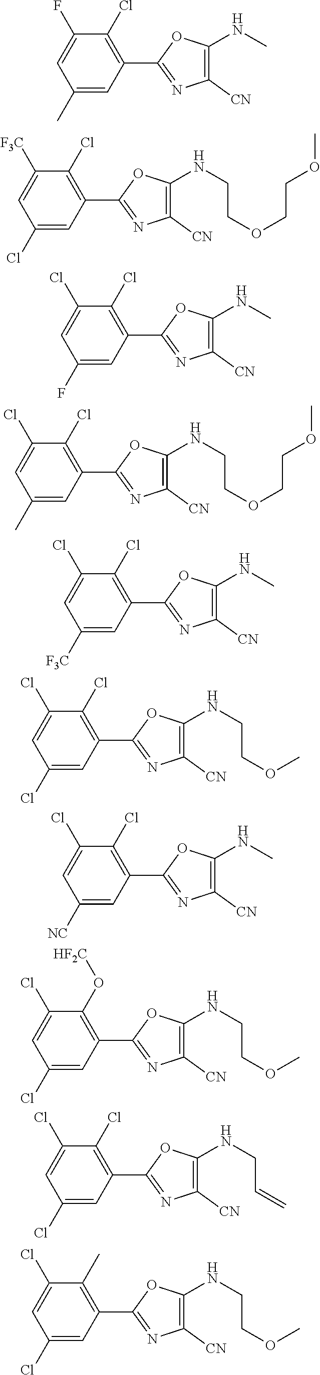

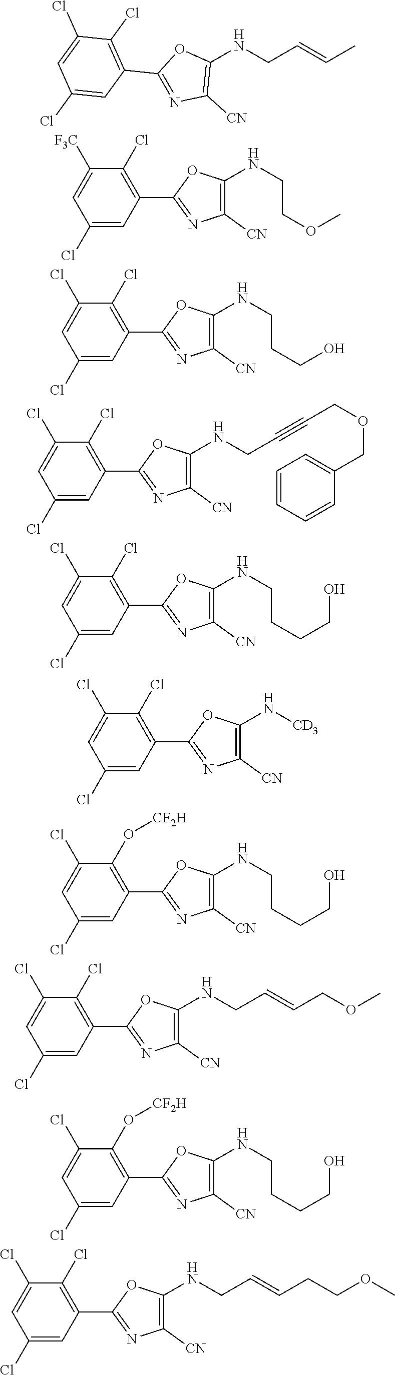

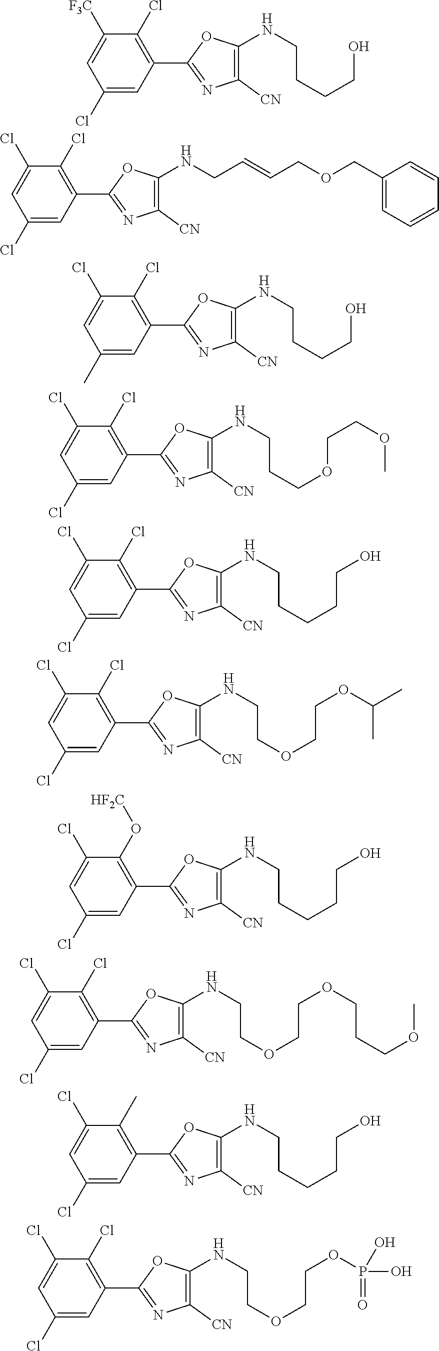

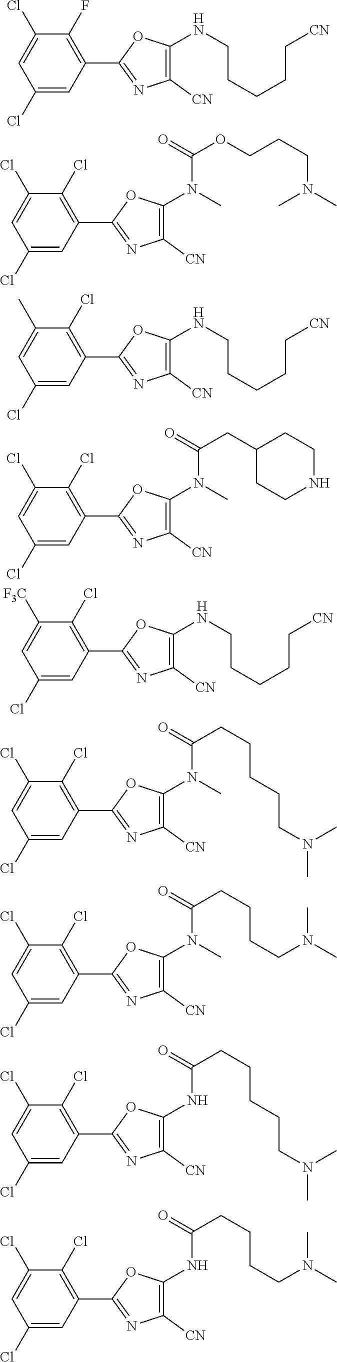

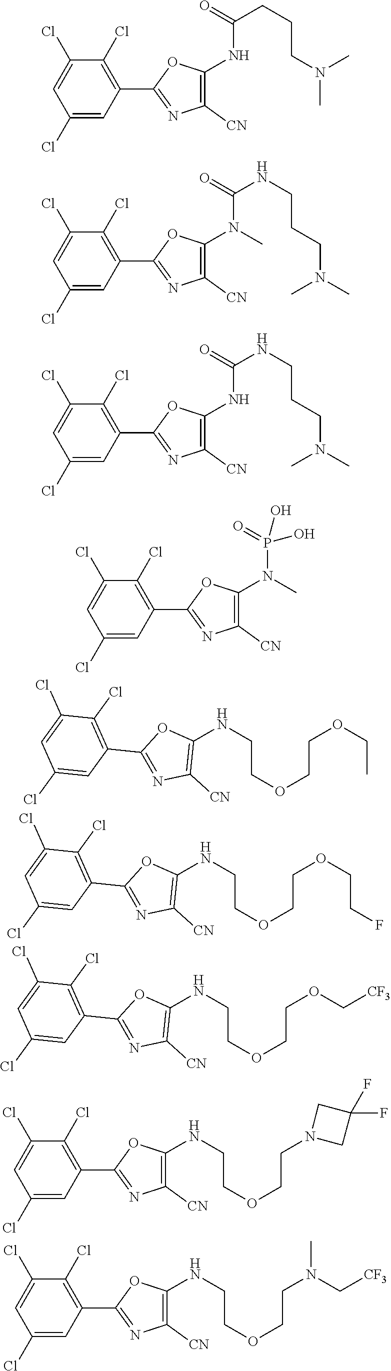

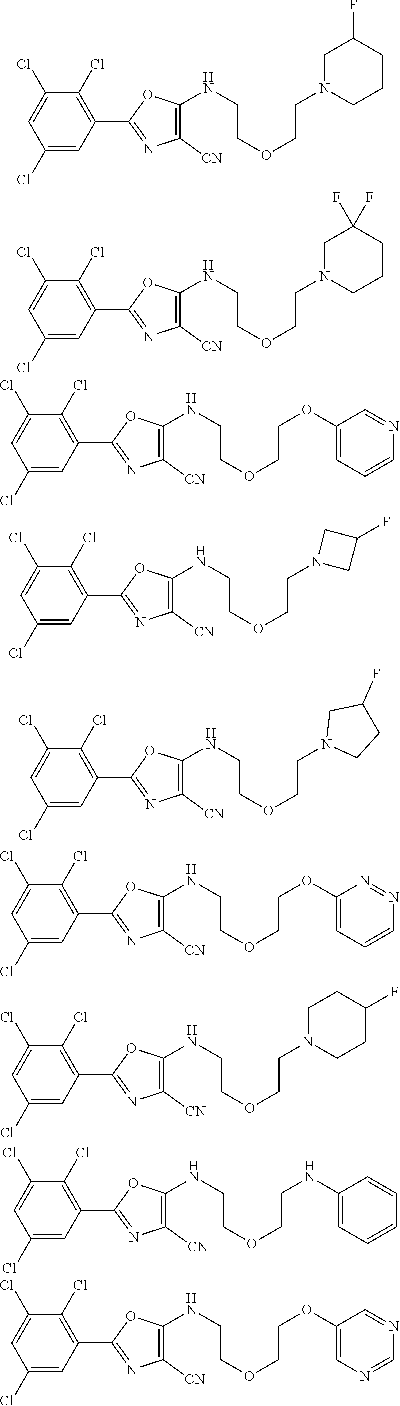

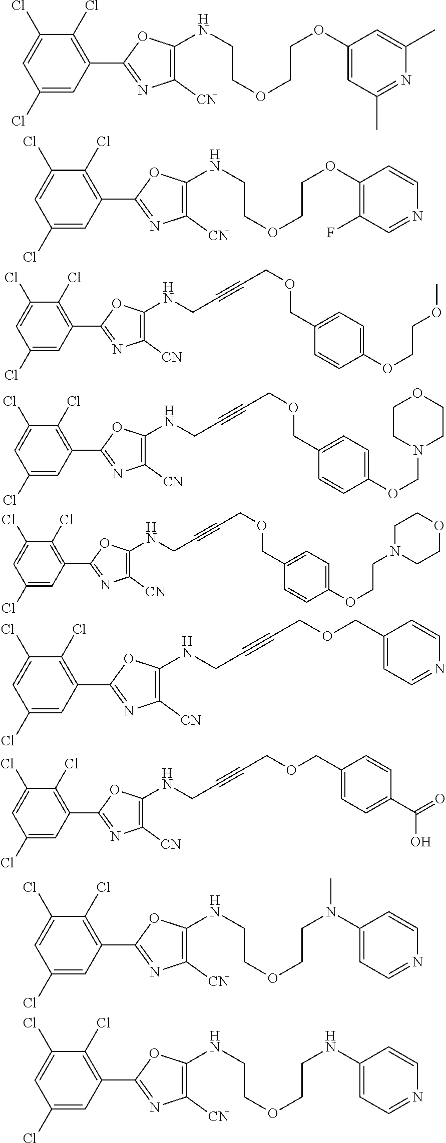

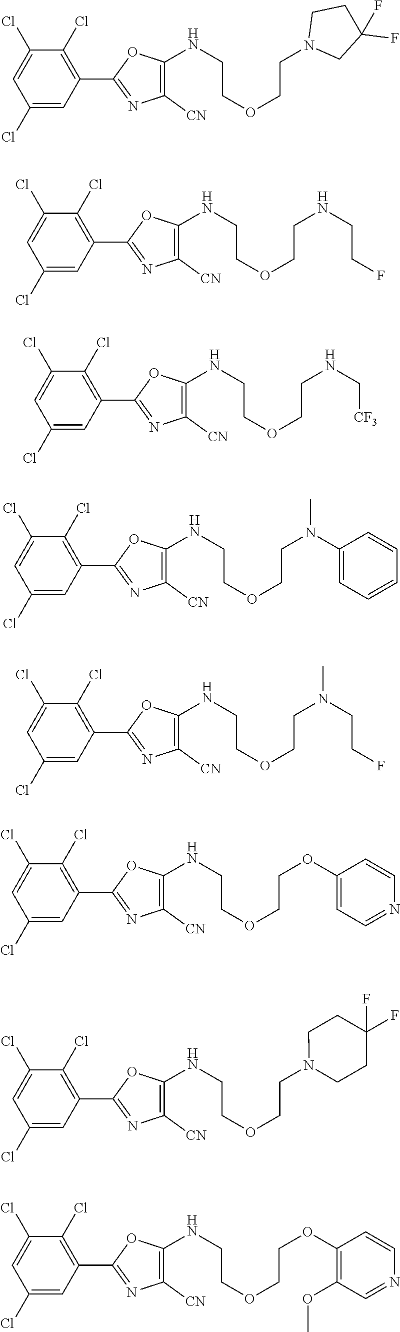

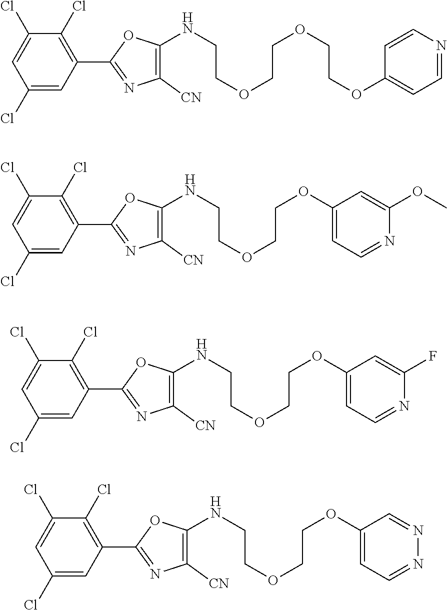

or a pharmaceutically acceptable salt thereof.
   32. The composition according to 30, wherein the lipoxygenase inhibitor is selected from the group consisting of:

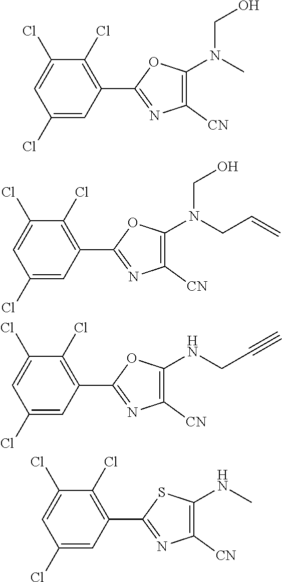

or a pharmaceutically acceptable salt thereof.
   33. The composition according to any one of 1-25, wherein the lipoxygenase inhibitor is a compound of Formula (III):

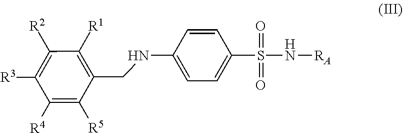

where R¹, R², R³, R⁴ and R⁵ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
R_{A} is cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl,
or a salt, solvate or hydrate thereof.
   34. The composition according to 33, wherein R_{A} is selected from the group consisting of 2-benzothiazole, 2-benzoxazole, 2-benzimidazole, 2-thiophene, 4-methyl-2-benzothiazole, 4-methyl-2-thiazole, 5-methyl-2-thiazole, 5-phenyl-2-thiazole, 4,5-methyl-2-thiazole, 5-methyl-3-isoxazole, 3-methoxy-phenyl, 3-quinolone, 8-isoquinolone, phenyl, 1-naphthalene, 2-naphthalene, 1,4-bi-phenyl, 1.3-bi-phenyl, 3-piperazine-phenyl, 4-piperazine-phenyl, 4-piperidine-phenyl, 4-piperazine-3-pyridine, 6-methyl-3-pyridine, 2-pyridine, 3-pyridine, 2-pyrimidine, 3-tert-butyl-phenyl, 6-methoxy-2-benzothiazole, 4-phenyl-2-thiazole, 3-morpholine-phenyl, 4N-boc-piperidine-3-phenyl, 3-piperidine-phenyl, 3-isopropyl-phenyl and 6-F-2-benzothiazole.
   35. The composition according to 33, wherein the lipoxygenase inhibitor is a compound of Formula (IIIB):

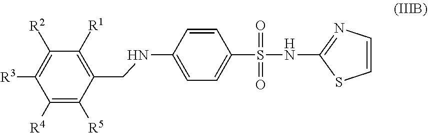

where R¹, R², R³, R⁴ and R⁵ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl.
   36. The composition according to 35, wherein the lipoxygenase inhibitor is a compound of Formula (IIIC):

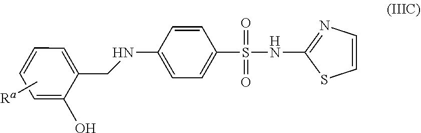

wherein R^{a} is selected from 2-amino-Ph; 3-OH-Ph; 2-amino-3-methoxy-Ph; 3-OH-4-methoxy-Ph; 7-indole; 2,3-Cl-Ph; 3-Cl; 3-F; 3-Br; 4-Br; 3-methyl; 3-amino; 3-nitro; 3-allyl; 4-Cl; 4-methoxy; 5-Cl; 5-methoxy; 5-nitro; 5-F; 5-nitro and 6-methoxy.
   37. The composition according to 35, wherein the lipoxygenase inhibitor is a compound of Formula (IIID):

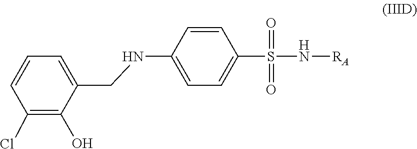

wherein R_{A} is selected from the group consisting of phenyl, 1-naphthalene, 2-benzothiazole, 2-naphthalene, 4-biphenyl, 8-isoquinoline, 3-quinoline and 4-piperidine-phenyl.
   38. The composition according to 35, wherein the lipoxygenase inhibitor is a compound of Formula (IIIE):

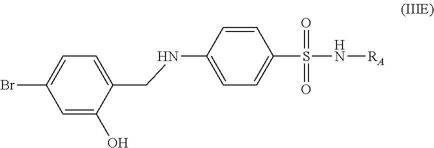

wherein R_{A} is selected from the group consisting of phenyl, 1-naphthalene, 2-benzothiazole, 3-quinoline, 2-naphthalene, 4-biphenyl and 8-quinoline.
   39. The composition according to 33, wherein the lipoxygenase inhibitor is a compound of Formula (IIIF):

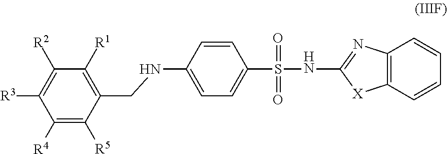

where R¹, R², R³, R⁴ and R⁵ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
X is S or O
or a salt, solvate or hydrate thereof.
   40. The composition according to 39, wherein the compound is selected from:

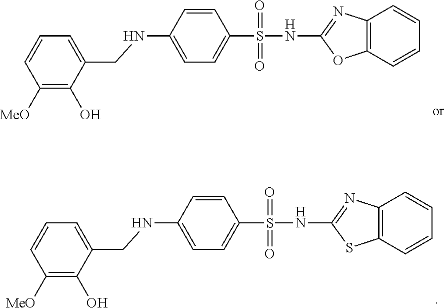 [SMILES: COc1cccc(CNc2ccc(S(=O)(=O)Nc3nc4ccccc4o3)cc2)c1O ; COc1cccc(CNc2ccc(S(=O)(=O)Nc3nc4ccccc4s3)cc2)c1O]

41. The composition according to any one of 1-25, wherein the lipoxygenase inhibitor is a compound of Formula (IV):

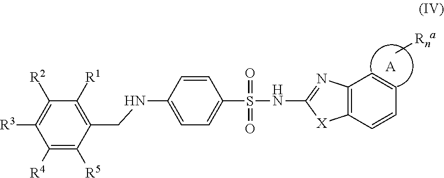

wherein R¹, R², R³, R⁴ and R⁵ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
X is S or O;
the A ring is a substituted or unsubstituted 5 to 12 membered ring;
n is an integer from 0 to 12; and
each R^{a} is independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl,
or a salt, solvate or hydrate thereof.
   42. The composition according to 41, wherein:
R¹ is hydroxy;
R² is alkoxy; and
each of R³, R⁴ and R⁵ are hydrogen.
   43. The composition according to any one of 41-42, wherein X is S.
   44. The composition according to any one of 41-43, wherein the compound is of formula IVA:

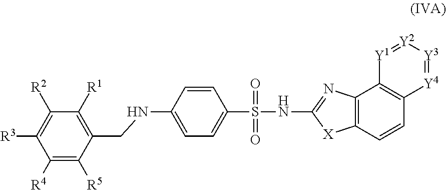

wherein Y₁, Y₂, Y₃ and Y₄ are each independently C or N.
   45. The composition according to 44, wherein:
Y₁, Y₂, Y₃ and Y₄ are each C; or
Y₃ is N; and Y₁, Y₂, and Y₄ are each C; or
Y₁ and Y₄ are N and Y₂ and Y₃ are C.
   46. The composition according to any one of 41-45, wherein:
A) A is unsubstituted; or
B) n is 1 or 2 and each R^{a} is independently selected from:

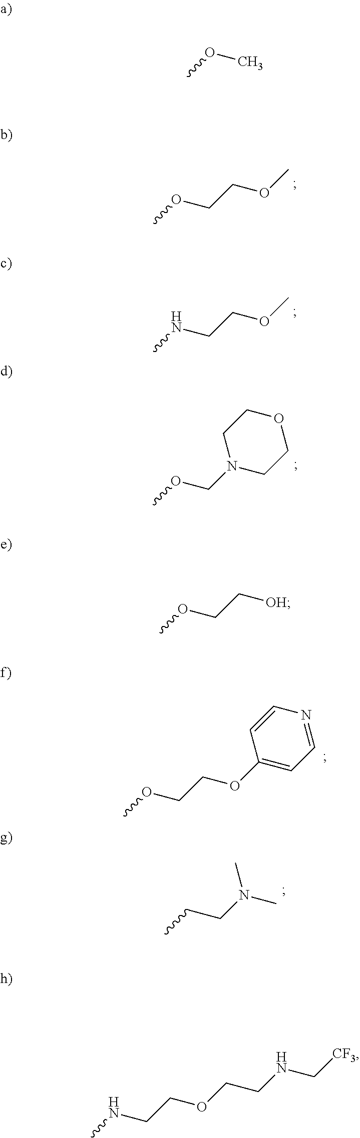

wherein [symbol] represents the A-R^{a} bond.
   47. The composition according to any one of 44-46, wherein the compound is selected from:

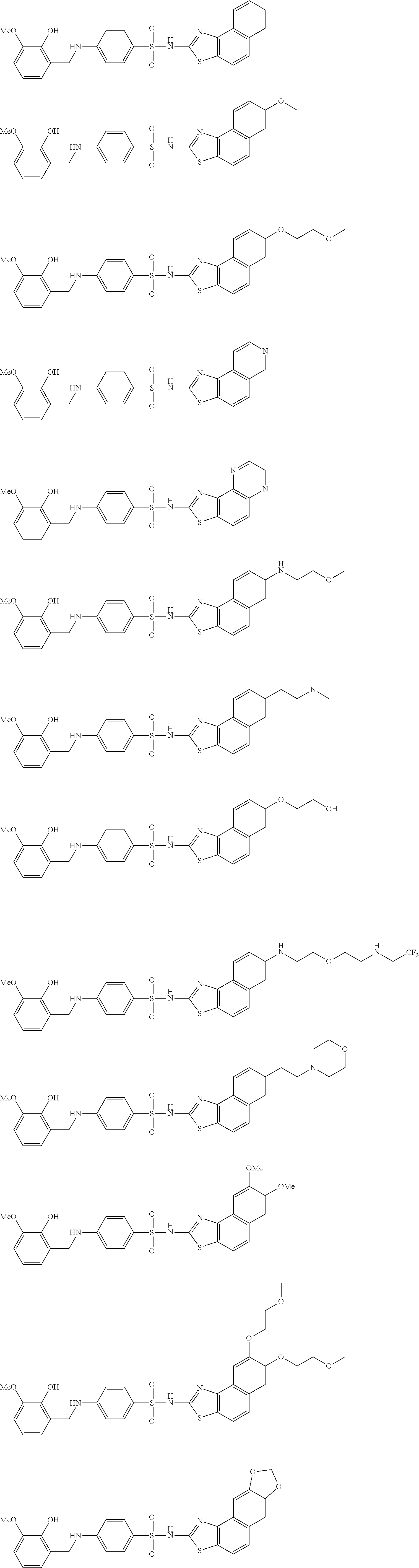

48. The composition according to any one of 41-43, wherein the compound is of formula IVB:

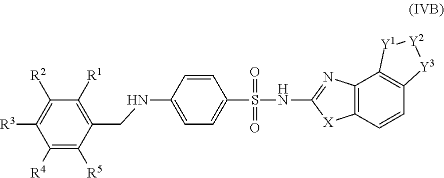

wherein Y¹, Y₂, Y₃ and Y₄ are each independently C, N or O.
   49. The composition according to 48, wherein:
Y₁, Y₂ and Y₃ are each C; or.

Y₂ is C and Y₁ and Y₃ are 0; or
Y₁ is N and Y₂ and Y₃ are C.
   50. The composition according to any one of 48-49, wherein:
A) A is unsubstituted; or
B) n is 1 or 2 and each R^{a} is independently selected from:

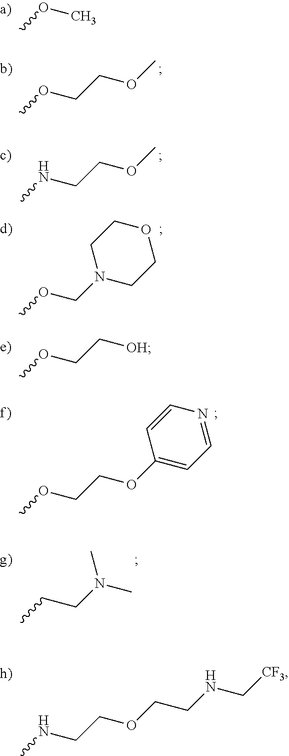

wherein [symbol] represents the A-R^{a} bond.
   51. The composition according to any one of 48-50, wherein the compound is selected from:

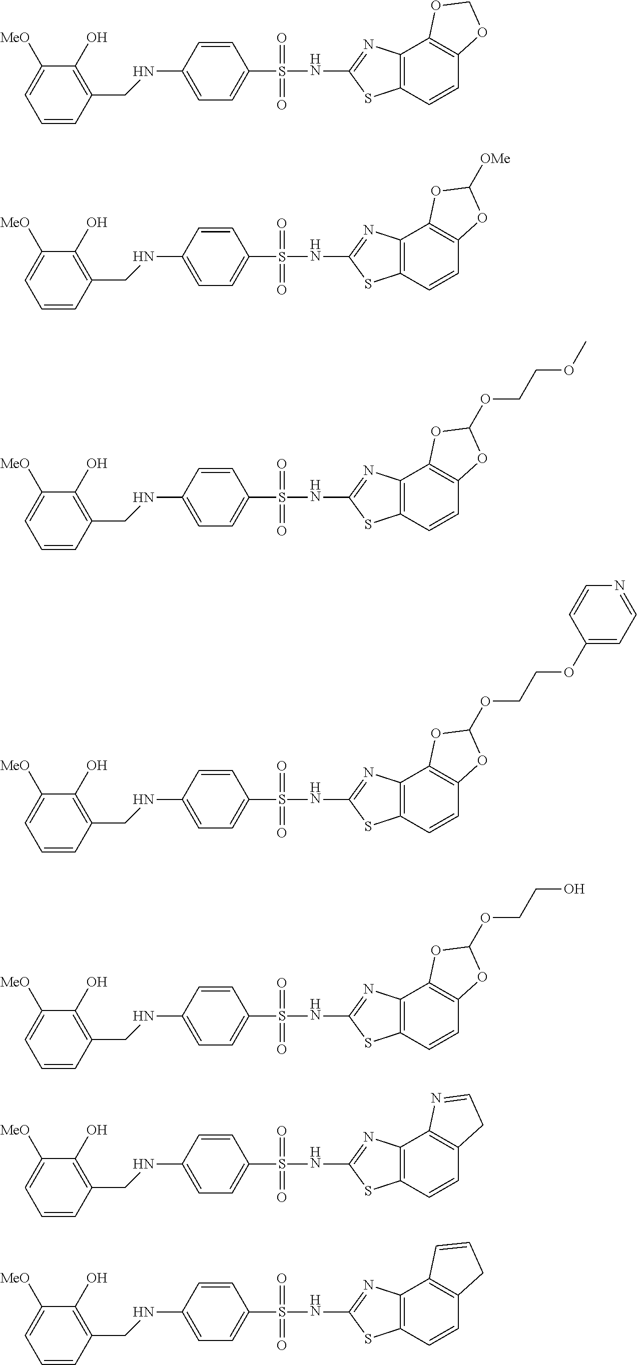

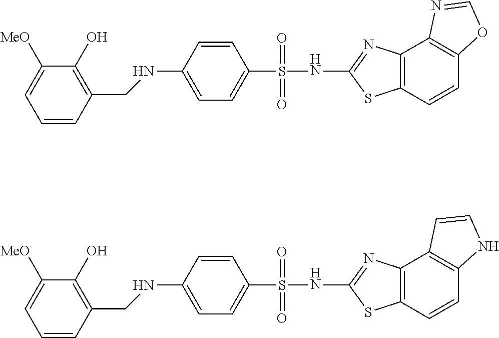 [SMILES: COc1cccc(CNc2ccc(S(=O)(=O)Nc3nc4c(ccc5ocnc54)s3)cc2)c1O ; COc1cccc(CNc2ccc(S(=O)(=O)Nc3nc4c(ccc5[nH]ccc54)s3)cc2)c1O]

52. 4-((2-hydroxy-3-methoxybenzyl)amino)-N-(naphtho[1,2-d]thiazol-2-yl)benzenesulfonamide:

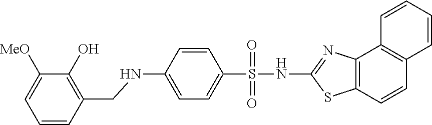 [SMILES: COc1cccc(CNc2ccc(S(=O)(=O)Nc3nc4c(ccc5ccccc54)s3)cc2)c1O]

or a pharmaceutically acceptable salt, solvate or hydrate thereof.
   53. A pharmaceutical composition comprising:
a composition according to any one of 1-52; and
a pharmaceutically acceptable excipient.
   54. A method comprising administering to a subject a therapeutically effective amount of a composition according to any one of 1-53.
   55. The method according to 54, wherein the composition is intravenously administered to the subject.
   56. The method according to 55, wherein the composition is intravenously administered to the subject by bolus injection.
   57. The method according to 55, wherein the composition is intravenously administered to the subject by sustained infusion.
   58. A method for treating or preventing a disease or disorder in which lipoxygenase (LOX) is implicated in the pathology, the method comprising administering to a subject a therapeutically effective amount of a composition according to any one of 1-53.
   59. The method according to 58, wherein the composition is intravenously administered to the subject.
   60. The method according to 58, wherein the composition is intravenously administered to the subject by bolus injection.
   61. The method according to 58, wherein the composition is intravenously administered to the subject by sustained infusion.
   62. The method according to any one of 54-61, wherein the subject is a subject that has or is diagnosed as having a disease or disorder selected from: stroke, diabetes, obesity, asthma, glomerulonephritis, osteoporosis, periventricular leukomalacia, cardiac arrest with resuscitation, atherosclerosis, neurodegenerative or neuroinflammatory disorders selected from Parkinson's disease, Alzheimer's disease, Huntington's disease and dementia, cancer, brain injury, a disease involving hypoxia or anoxia, myocardial infarction, cardiovascular disease, chronic heart failure, congestive heart, ischemia selected from cerebral ischemia, retinal ischemia, myocardial ischemia, or post-surgical cognitive dysfunction, inflammatory disease, arterial inflammation, inflammatory bowel disease, Crohn's disease, renal disease, asthma, allergic rhinitis, gout, cardiopulmonary inflammation, rheumatoid arthritis, osteoarthritis, muscle fatigue, acne, dermatitis, or psoriasis), chronic bronchitis, mucus hypersecretion, chronic obstructive pulmonary disease (COPD), pulmonary fibrosis, fibrosis caused by chemotherapy, idiopathic pulmonary fibrosis, cystic fibrosis, adult respiratory distress syndrome, CNS disorders, psychiatric disorders, anxiety, depression, peripheral neuropathy, spinal cord injury, head injury, surgical trauma, allograft tissue or organ transplant rejection, autoimmune disorder, eczema and disorders involving bone loss or bone formation.
   63. The method according to 62, wherein the subject is diagnosed with a cardiovascular disease.
   64. The method according to 63, wherein the subject is diagnosed with atherosclerosis.
   65. The method according to any one of 62-64, wherein the subject is diagnosed with a stroke.
   66. The method according to 62, wherein the subject is diagnosed with cystic fibrosis.
   67. The method according to 62, wherein the subject is diagnosed with a neurodegenerative disease.
   68. The method according to 67, wherein the subject is diagnosed with Alzheimer's disease.
   69. The method according to 67, wherein the subject is diagnosed with Parkinson's disease.
   70. The method according to 67, wherein the subject is diagnosed with Huntington's disease.
   71. A method for inhibiting a human epithelial lipoxygenase, the method comprising contacting a cell with a composition according to any one of 1-53.
   72. The method according to 71, wherein the human epithelial lipoxygenase comprises 12/15-lipoxygenase.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for.

Compositions were generally prepared by mixing with solvent and sonicated at room temperature in a water bath for 10 minutes. After a 15 minute waiting period at room temperature, the composition was sonicated again for 15 minutes at room temperature. Table 1 provides a list of formulations prepared for Compound 27332 according to certain embodiments. Table 2 provides a list of formulations prepared for 5-(methylamino)-2-naphthalen-1-yl-1,3-oxazole-4-carbonitrile (Compound ML351) according to certain embodiments. Compositions were analyzed by absorbance spectrometry at 340 nm to characterize the solubility of the compounds in each solvent mixture. Measurements were taken using a Cary60 spectrophotometer. Briefly, 2 ml absolute EtOH was added to cuvette, and instrument blanked. Then 2 μl of the excipient buffer was added, and scanned from 450 nm to 200 nm in order to confirm no large/interfering peak at 340 nm. Next, 2 μl of 1.10 diluted (in absolute EtOH) was added to the cuvette and scanned (450 nm to 200 nm)—this was repeated twice more for a total of three measurements. The difference in Abs(340) was determined for each subsequent addition. These three measurements were used to calculate the concentration (in molarity) using previously determined epsilon, and converted back to mg/ml.

TABLE 1
Compositions of Compound 27332

| | | | | | | | | Soluble after |
|---|---|---|---|---|---|---|---|---|
| | | Approx | | | Measured | | | 24 hours at |
| | | Conc. | Average | Std | Conc. | Std | | Room |
| Solvent Composition | Soluble | (mg/ml) | Abs340 | Dev | (mg/ml) | Dev | Viscosity | Temperature |
| 10/90 solutol/PEG400 | yes | 30 | N/D | N/D | N/D | N/D | medium | N/D |
| 10/90 solutol/PEG400 | no | 50 | 0.0131 | 0.0018 | 27.0 | 3.7 | medium | no |
| 20/80 DMA/PEG400 | yes | 50 | | | | | low | |
| 20/80 DMA/PEG400 | yes | 75.5 | 0.4048 | 0.0242 | 83.1 | 5.0 | low | |
| 20/80 DMA/PEG400 | yes | 94.2 | 0.4013 | 0.0098 | 82.4 | 2.0 | low | |
| 20/80 DMA/PEG400 | yes | 50.0 | 0.2753 | 0.0111 | 56.5 | 2.3 | low | yes |
| 20/80 DMA/PEG400 | yes | 90.7 | 0.2553 | 0.0130 | 104.8 | 5.3 | low | yes |
| 20/80 DMA/PEG400 | yes | 90.7 | 0.2247 | 0.0125 | 92.2 | 5.1 | low | |
| 15/85 DMA/PEG400 | yes | 50 | 0.2613 | 0.0270 | 53.6 | 5.5 | low | yes |
| 15/85 DMA/PEG400 | yes | 63.5 | 0.1725 | 0.0077 | 70.8 | 3.2 | low | yes |
| 15/85 DMA/PEG400 | yes | 81.0 | 0.2152 | 0.0044 | 88.3 | 1.8 | | |
| 15/85 DMA/PEG400 | yes | 81.0 | 0.2130 | 0.0016 | 87.4 | 0.7 | low | |
| 10/90 DMA/PEG400 | yes | 50 | 0.2850 | 0.0197 | 58.5 | 4.1 | low | yes |
| 10/90 DMA/PEG400 | yes | 69.6 | 0.1829 | 0.0108 | 75.1 | 4.4 | | yes |
| 10/90 DMA/PEG400 | yes | 85.6 | 0.1793 | 0.0044 | 73.6 | 1.8 | low | |
| 10/90 DMA/PEG400 | yes | 85.6 | 0.1635 | 0.0088 | 67.1 | 3.6 | | |
| 10/10/80 | yes | 50 | | | | | medium | |
| DMA/EtOH/PEG400 | | | | | | | | |
| 10/10/80 | no | 60.5 | 0.2910 | 0.0024 | 59.7 | 0.5 | medium | no |
| DMA/EtOH/PEG400 | | | | | | | | |
| 10/10/80 | yes | 50.0 | 0.3186 | 0.0062 | 65.4 | 1.3 | | |
| DMA/EtOH/PEG400 | | | | | | | | |
| 10/10/40/40 | no | 50 | 0.1340 | 0.0246 | 2.8 | 0.5 | high | no |
| DMA/EtOH/PEG400/H₂O | | | | | | | | |
| 20/80 DMSO/PEG400 | yes | 50 | 0.2588 | 0.0198 | 53.1 | 4.1 | low | yes |
| 20/80 DMSO/PEG400 | yes | 77.5 | 0.1993 | 0.0230 | 81.8 | 9.5 | low | yes |
| 20/80 DMSO/PEG400 | yes | 129.6 | 0.2327 | 0.0024 | 95.5 | 1.0 | | |
| 20/80 DMSO/PEG400 | yes | 94.8 | 0.2774 | 0.1639 | 113.9 | 67.3 | low | |
| 15/85 DMSO/PEG400 | yes | 50 | 0.2615 | 0.0081 | 53.7 | 1.7 | low | yes |
| 15/85 DMSO/PEG400 | yes | 64.3 | 0.1664 | 0.0208 | 68.3 | 8.5 | low | yes |
| 15/85 DMSO/PEG400 | yes | 77.7 | 0.1696 | 0.0119 | 69.6 | 4.9 | | |
| 15/85 DMSO/PEG400 | yes | 77.7 | 0.1839 | 0.0091 | 75.5 | 3.7 | low | |
| 20/80 | yes | 50.0 | 0.1434 | 0.0075 | 58.8 | 3.1 | low | |
| Diethylaetamide/PEG400 | | | | | | | | |
| 20/80 | yes | 50 | 0.1293 | 0.0012 | 53.1 | 0.5 | low | |
| Diethylaetamide/PEG400 | | | | | | | | |
| intralipid with 27332 | yes | 0.250 | 0.2014 | 0.01117 | 0.33065 | 0.01834 | low | |
| 10/90 Kolliphor | yes | 50 | | | | | medium | |
| EL/PEG400 | | | | | | | | |
| 10/90 Kolliphor | no | 59.5 | 0.1882 | 0.0076 | 38.6 | 1.6 | medium | no |
| EL/PEG400 | | | | | | | | |
| 10/90 Kolliphor | yes | 50.0 | 0.4482 | 0.0143 | 92.0 | | | |
| EL/PEG400 (AMRI) | | | | | | | | |
| 10/90 Kolliphor | no | 50.0 | 0.2746 | 0.0109 | 56.4 | | | |
| EL/PEG400 (BASF) | | | | | | | | |
| 50/50 Kolliphor EL/EtOH | no | 50 | 0.0390 | 0.0045 | 8.0 | 0.9 | high | no |
| 10/90 Kolliphor | yes | 50 | 0.1782 | 0.0106 | 36.6 | 2.2 | medium | |
| RH40/PEG400 | | | | | | | high | |
| 10/90 Kolliphor | no | 50.7 | 0.2269 | 0.0031 | 46.6 | 0.6 | medium | no |
| RH40/PEG400 | | | | | | | high | |
| 10/90 Kolliphor P188 | no | 50 | 0.0541 | 0.0021 | 11.1 | 0.4 | very high | no |
| Geismar/PEG400 | | | | | | | | |
| 10/90 Soluplus/PEG400 | yes | 50 | 0.1078 | 0.0040 | 22.1 | 0.8 | high | |
| 10/90 Soluplus/PEG400 | yes | 45.9 | 0.1355 | 0.0107 | 27.8 | 2.2 | high | |
| 10/90 Capryol/PEG400 | yes | 50 | 0.2010 | 0.0018 | 41.3 | 0.4 | low | |
| 10/90 Capryol/PEG400 | yes | 54.1 | 0.2973 | 0.0075 | 61.0 | 1.5 | low | |
| 10/90 Capryol/PEG400 | no | 50.0 | 0.3269 | 0.0131 | 67.1 | 2.7 | | |
| 10/90 Transcutol/PEG400 | yes | 50 | 0.2268 | 0.0034 | 46.5 | 0.7 | low | |
| 10/90 Transcutol/PEG400 | | 55.9 | 0.2825 | 0.0302 | 58.0 | 6.2 | | |
| 10/90 Transcutol/PEG400 | yes | 50.0 | 0.3159 | 0.0087 | 64.8 | 1.8 | low | no |
| 10/90 Labrafil/PEG400 | no | 50 | 0.1718 | 0.0080 | 35.3 | 1.6 | medium | no |
| 10/90 Labrasol/PEG400 | no | 50 | 0.1985 | 0.0074 | 40.7 | 1.5 | medium | no |
| 10/90 | no | 50 | 0.2060 | 0.0027 | 42.3 | 0.6 | medium | no |
| Lauroglycol/PEG400 | | | | | | | | |
| 40% Captisol in H2O | no | 50 | 0.2497 | 0.0145 | 10.2 | 0.6 | med high | no |
| 40% Kleptose in H2O | no | 50 | 0.0131 | 0.0018 | 0.5 | 0.1 | high | no |

TABLE 2
Compositions of 5-(methylamino)-2-naphthalen-1-yl-1,3-oxazole-4-carbonitrile (Compound ML351)

| | | | | | | | | Soluble after |
|---|---|---|---|---|---|---|---|---|
| | | Approx | | | Measured | | | 24 hours at |
| | | Conc. | Average | Std | Conc. | Std | | Room |
| Solvent Composition | Soluble | (mg/ml) | Abs340 | Dev | (mg/ml) | Dev | Viscosity | Temperature |
| 20/80 DMA/PEG400 | yes | 50 | 0.3544 | 0.0208 | 46.1 | 2.7 | low | yes |
| 20/80 DMA/PEG400 | yes | 78.1 | 0.2909 | 0.0195 | 75.7 | 5.1 | low | yes |
| 20/80 DMA/PEG400 | No | 90.5 | 0.2099 | 0.0074 | 66.3 | 2.3 | low | |
| 20/80 DMA/PEG400 | No | 90.5 | 0.1738 | 0.0184 | 54.9 | 5.8 | low | |
| 20/80 | yes | 50.0 | 0.1663 | 0.0279 | 52.5 | 8.8 | low | |
| Diethylacetamide/PEG400 | | | | | | | | |
| 20/80 | yes | 50 | 0.1440 | 0.0028 | 45.5 | 0.9 | low | |
| Diethylacetamide/PEG400 | | | | | | | | |
| Intralipid with ML351 | yes | 0.250 | 0.2494 | 0.01432 | 0.31500 | 0.01809 | low | |

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a feature in the claim only when the exact phrase “means for” or the exact phrase “step for” is recited at the beginning of such feature in the claim; if such exact phrase is not used in a feature in the claim, then 35 U.S.C. § 112(f) or 35 U.S.C. § 112(6) is not invoked.

What is claimed is:

1. A composition comprising:
a solvent component comprising:
a first solvent selected from the group consisting of dimethylacetamide (DMA), diethylacetamide (DEA), dimethylsulfoxide (DMSO), a polyoxyethylene ester of 12-hydroxysteraric acid, a polyethoxylated castor oil, a polyvinyl caprolactam-polyvinyl acetate-polyethylene glycol, a propylene glycol caprylate, a diethylene glycol monomethyl ether, a caprylocaproyl macrogolglyceride, a polyoxyl-6 glyceride and a propylene glycol monolaurate; and
a second solvent comprising a polyethylene glycol; and
a lipoxygenase inhibitor,
wherein the lipoxygenase inhibitor is present in the composition at a concentration of 30 mg/mL or greater.

2. The composition according to claim 1, wherein the first solvent and second solvent are present in the composition at a ratio of from 5:95 to 50:50.

3. The composition according to any one of claims 1-2, wherein the lipoxygenase inhibitor is present in the composition at a concentration of 50 mg/mL or more.

4. The composition according to any one of claims 1-3, wherein the lipoxygenase inhibitor is a compound of Formula (I): 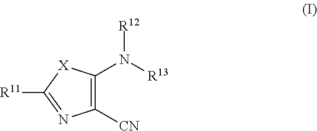
or a pharmaceutically acceptable salt thereof, in which:
X is O or S;
R¹¹ is an aryl, heteroaryl, cyclyl, or heterocyclyl, each of which can be optionally substituted; and
R¹² and R¹³ are independently hydrogen, halogen, alkyl, alkenyl, alkynyl, aralkyl, acyl, aryl, heteroaryl, cyclyl or heterocyclyl, each of which can be optionally substituted
or a pharmaceutically acceptable salt thereof.

5. The composition according to claim 4, wherein the lipoxygenase inhibitor is a compound of Formula (Ia): 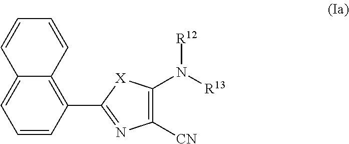
or a pharmaceutically acceptable salt thereof, in which:
X is O or S;
R¹² and R¹³ are independently hydrogen, halogen, alkyl, alkenyl, alkynyl, aralkyl, acyl, aryl, heteroaryl, cyclyl or heterocyclyl, each of which can be optionally substituted
or a pharmaceutically acceptable salt thereof.

6. The composition according to claim 5, wherein the compound is selected from the group consisting of: 5-(methylamino)-2-naphthalen-1-yl-1,3-oxazole-4-carbonitrile (ML351), 2-(2,3-dichlorophenyl)-5-(methylamino)-1,3-oxazole-4-carbonitrile, 2-(3,4-dichlorophenyl)-5-(methylamino)-1,3-oxazole-4-carbonitrile, 5-(methylamino)-2-naphthalen-1-yl-1,3-thiazole-4-carbonitrile or a pharmaceutically acceptable salt thereof.

7. The composition according to any one of claims 1-3, wherein the lipoxygenase inhibitor is a compound of Formula (II): 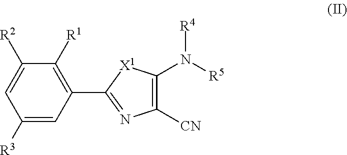
or a pharmaceutically acceptable salt thereof, wherein:
X¹ is selected from O and S;
R¹, R², and R³ are each independently selected from halo, CN, C₁₋₃ alkyl, C₁₋₃ haloalkyl, C₁₋₃ alkoxy, and C₁₋₃ haloalkoxy;
R⁴ is selected from H, C₁₋₃ alkyl, and HO—C₁₋₃ alkylene;
R⁵ is selected from C₁₋₆ alkyl, C₂₋₆ alkenyl, C₂₋₆ alkynyl, C(O)OR^{a1},
C(O)N(R^{a1})₂, P(═O)(OR^{a1})₂, and C(O)R¹; wherein said C₁₋₆ alkyl, C₂₋₆ alkenyl, and C₂₋₆ alkynyl are each optionally substituted with a substituent selected from OR^{a1} and OP(═O)(OR^{a1})₂;
each R^{a1} is independently selected from H, C₁₋₆ alkyl, C₆₋₁₀ aryl, C₁₋₆ alkyl-C₆₋₁₀ aryl, and C₁₋₆ alkyl-C₆₋₁₀ aryl-C₁₋₆ alkyl, wherein said C₁₋₆ alkyl, C₆₋₁₀ aryl, C₁₋₆ alkyl-C₆₋₁₀ aryl, and C₁₋₆ alkyl-C₆₋₁₀ aryl-C₁₋₆ alkyl are each optionally substituted with a substituent selected from amino, C₁₋₆ alkylamino, (C₁₋₆ haloalkyl)amino, di(C₁₋₆ alkyl)amino, (C₁₋₆ alkyl)(C₁₋₆ haloalkyl)amino, (C₆₋₁₀ aryl)amino, (C₆₋₁₀ aryl)(C₁₋₆ alkyl)amino, (5-6-membered heteroaryl)amino, (5-6-membered heteroaryl)(C₁₋₆ alkyl)amino, C₆₋₁₀ aryl, 4-6 membered heterocycloalkyl, 5-6-membered heteroaryl, and OR^{a2}, wherein said C₆₋₁₀ aryl, 4-6 membered heterocycloalkyl, and 5-6-membered heteroaryl are each optionally substituted with 1, 2, or 3 substituents independently selected from amino, C₁₋₆ alkylamino, di(C₁₋₆ alkyl)amino, carboxy, and halo;
each R^{a2} is independently selected from H, C₁₋₃ alkyl, C₁₋₃ haloalkyl, C₁₋₃ alkoxy-C₁₋₃ alkyl, 4-7 membered heterocycloalkyl-C₁₋₃ alkyl, 5-6-membered heteroaryloxy-C₁₋₃ alkyl, C₆₋₁₀ aryl, and 5-6-membered heteroaryl, wherein said C₆₋₁₀ aryl and 5-6-membered heteroaryl are each optionally substituted with 1, 2, or 3 substituents independently selected from halo, C₁₋₃ alkoxy, C₁₋₃ haloalkoxy, C₁₋₃ alkyl, and C₁₋₃ haloalkyl; and
R^{b1} is C₁₋₆ alkyl, optionally substituted with a substituent selected from amino, C₁₋₆ alkylamino, di(C₁₋₆ alkyl)amino, and 4-7 membered heterocycloalkyl ring comprising at least one N atom,
or a pharmaceutically acceptable salt thereof.

8. The composition according to claim 7, wherein the lipoxygenase inhibitor is selected from the group consisting of 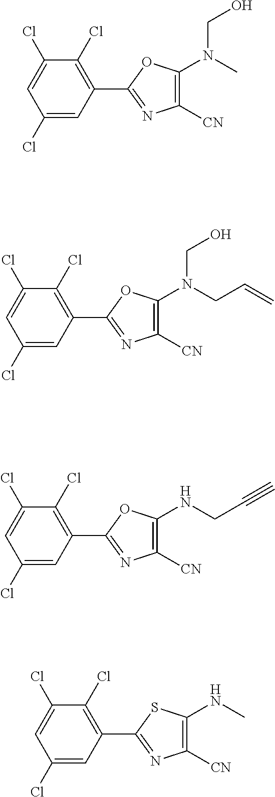 [SMILES: CN(CO)c1oc(-c2cc(Cl)cc(Cl)c2Cl)nc1C#N ; C=CCN(CO)c1oc(-c2cc(Cl)cc(Cl)c2Cl)nc1C#N ; C#CCNc1oc(-c2cc(Cl)cc(Cl)c2Cl)nc1C#N ; CNc1sc(-c2cc(Cl)cc(Cl)c2Cl)nc1C#N]
or a pharmaceutically acceptable salt thereof.

9. The composition according to any one of claims 1-3 wherein the lipoxygenase inhibitor is a compound of Formula (III): 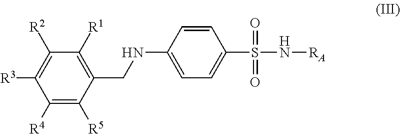
where R¹, R², R³, R⁴ and R⁵ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
R_{A} is cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl,
or a salt, solvate or hydrate thereof.

10. The composition according to claim 9, wherein R_{A} is selected from the group consisting of 2-benzothiazole, 2-benzoxazole, 2-benzimidazole, 2-thiophene, 4-methyl-2-benzothiazole, 4-methyl-2-thiazole, 5-methyl-2-thiazole, 5-phenyl-2-thiazole, 4,5-methyl-2-thiazole, 5-methyl-3-isoxazole, 3-methoxy-phenyl, 3-quinolone, 8-isoquinolone, phenyl, 1-naphthalene, 2-naphthalene, 1,4-bi-phenyl, 1.3-bi-phenyl, 3-piperazine-phenyl, 4-piperazine-phenyl, 4-piperidine-phenyl, 4-piperazine-3-pyridine, 6-methyl-3-pyridine, 2-pyridine, 3-pyridine, 2-pyrimidine, 3-tert-butyl-phenyl, 6-methoxy-2-benzothiazole, 4-phenyl-2-thiazole, 3-morpholine-phenyl, 4N-boc-piperidine-3-phenyl, 3-piperidine-phenyl, 3-isopropyl-phenyl and 6-F-2-benzothiazole.

11. The composition according to claim 9, wherein the compound is selected from:

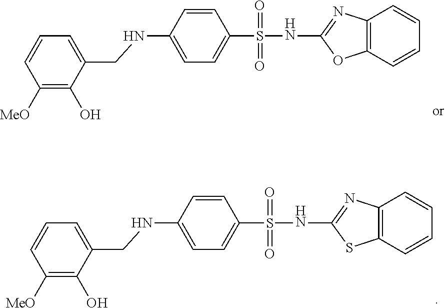 [SMILES: COc1cccc(CNc2ccc(S(=O)(=O)Nc3nc4ccccc4o3)cc2)c1O ; COc1cccc(CNc2ccc(S(=O)(=O)Nc3nc4ccccc4s3)cc2)c1O]

12. The composition according to any one of claims 1-3, wherein the lipoxygenase inhibitor is a compound of Formula (IV): 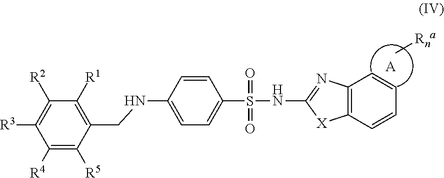
wherein R¹, R², R³, R⁴ and R⁵ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
X is S or O;
the A ring is a substituted or unsubstituted 5 to 12 membered ring;
n is an integer from 0 to 12; and
each R^{a} is independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl,
or a salt, solvate or hydrate thereof.

13. The composition according to claim 12, wherein the compound is selected from:

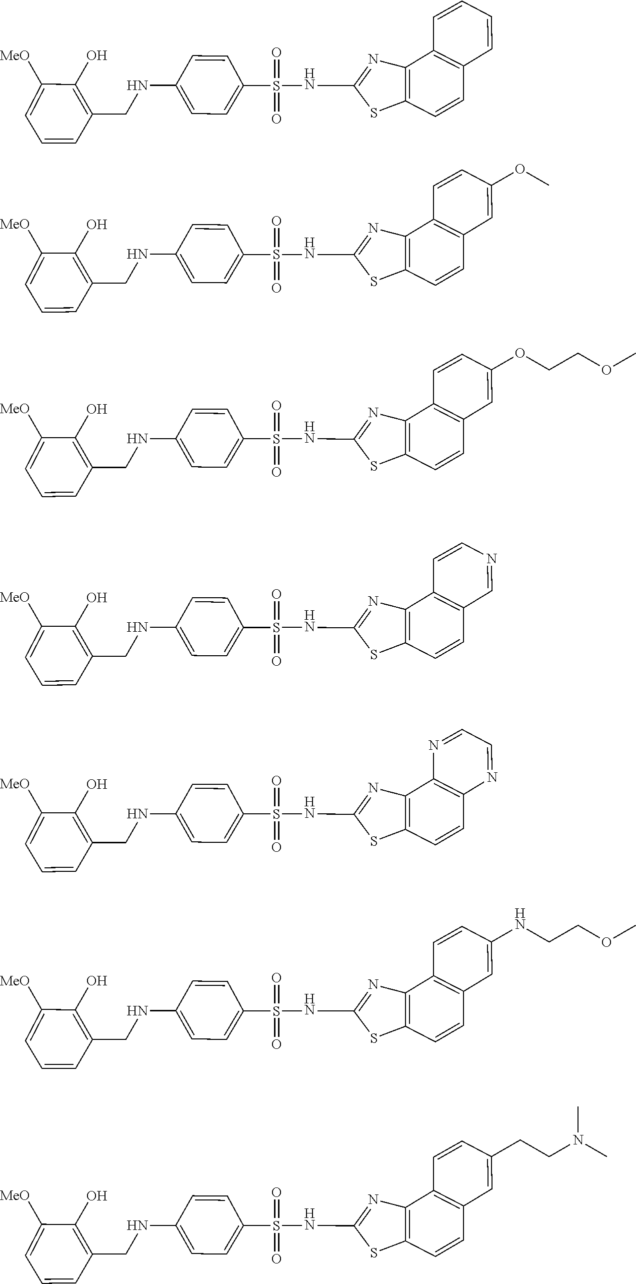

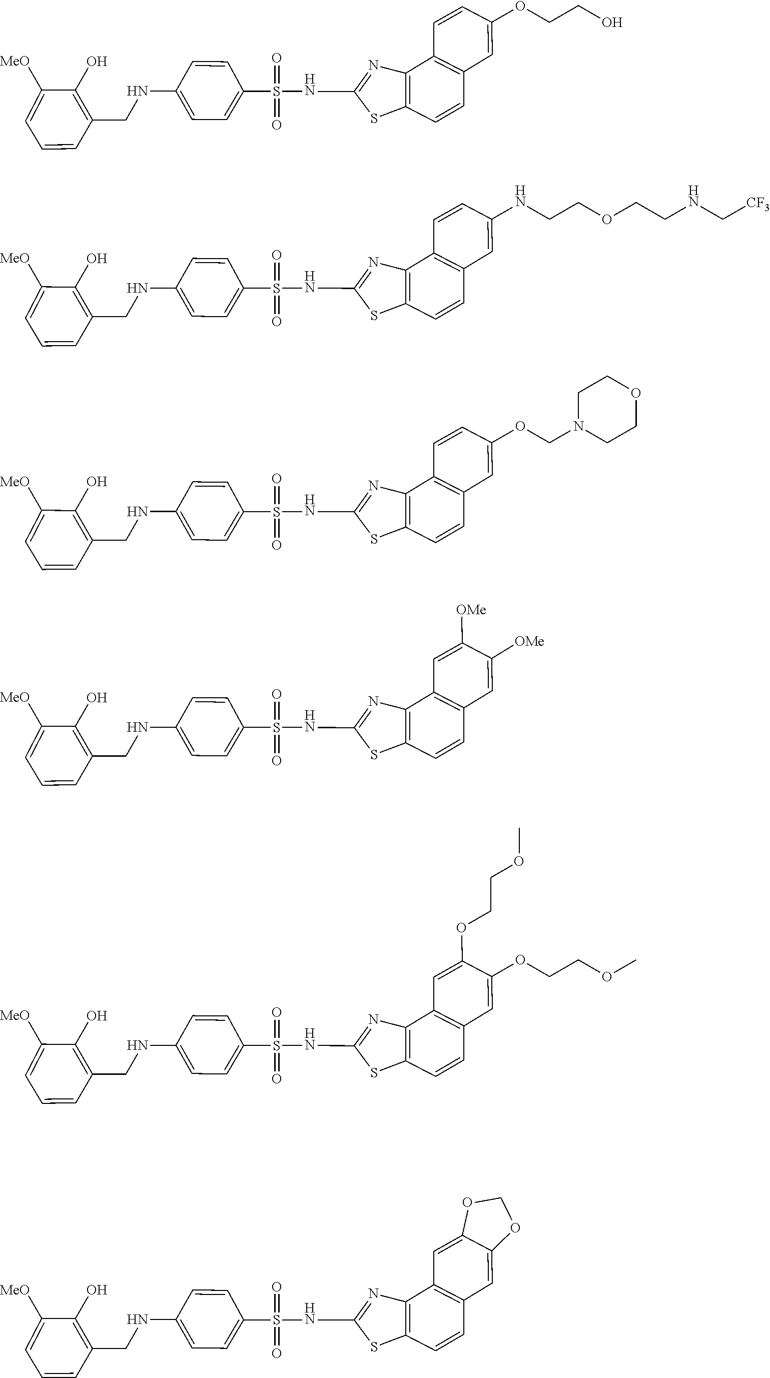 [SMILES: COc1cccc(CNc2ccc(S(=O)(=O)Nc3nc4c(ccc5cc(OCCO)ccc54)s3)cc2)c1O ; COc1cccc(CNc2ccc(S(=O)(=O)Nc3nc4c(ccc5cc(NCCOCCNCC(F)(F)F)ccc54)s3)cc2)c1O ; COc1cccc(CNc2ccc(S(=O)(=O)Nc3nc4c(ccc5cc(OCN6CCOCC6)ccc54)s3)cc2)c1O ; COc1cc2ccc3sc(NS(=O)(=O)c4ccc(NCc5cccc(OC)c5O)cc4)nc3c2cc1OC ; COCCOc1cc2ccc3sc(NS(=O)(=O)c4ccc(NCc5cccc(OC)c5O)cc4)nc3c2cc1OCCOC ; COc1cccc(CNc2ccc(S(=O)(=O)Nc3nc4c(ccc5cc6c(cc54)OCO6)s3)cc2)c1O]

14. The composition according to claim 12, wherein the compound is selected from:

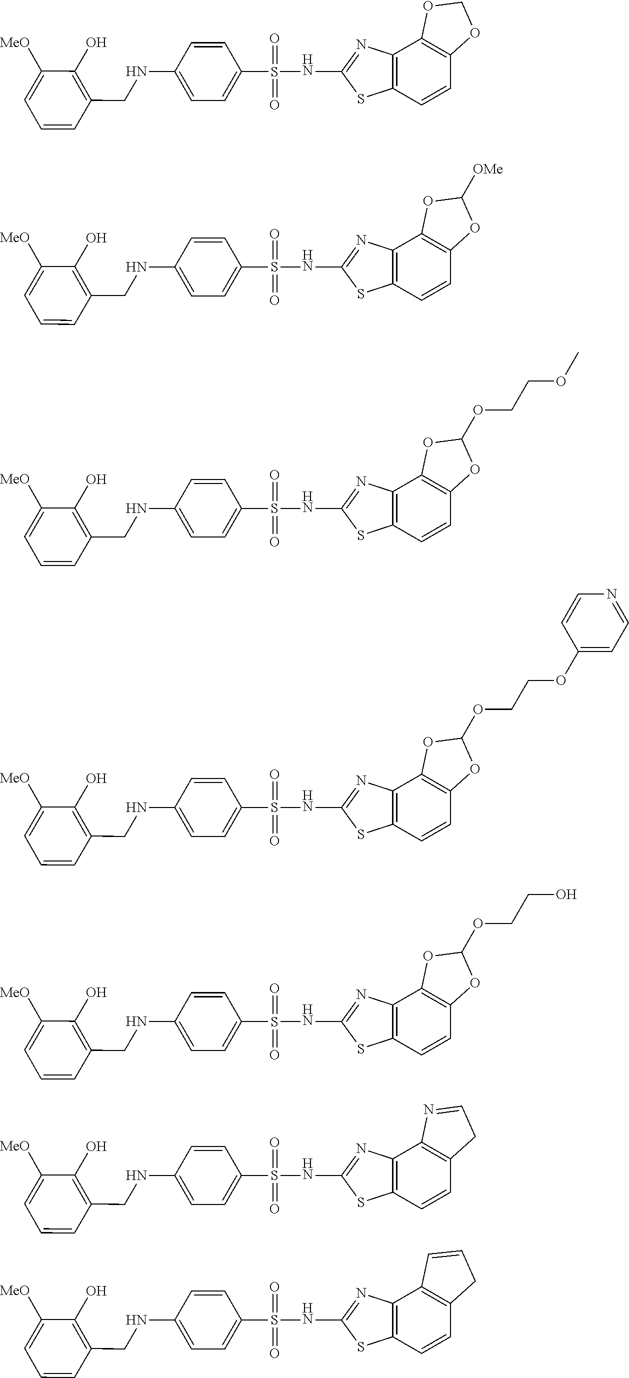

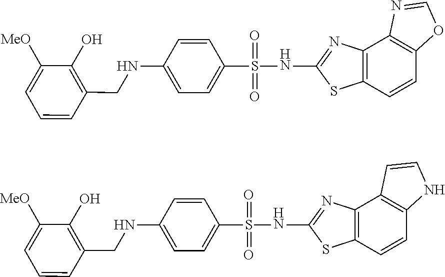 [SMILES: COc1cccc(CNc2ccc(S(=O)(=O)Nc3nc4c(ccc5ocnc54)s3)cc2)c1O ; COc1cccc(CNc2ccc(S(=O)(=O)Nc3nc4c(ccc5[nH]ccc54)s3)cc2)c1O]

15. A pharmaceutical composition comprising:
a composition according to any one of claims 1-14; and
a pharmaceutically acceptable excipient.
